(19) United States
(12) Patent Application Publication
(10) Pub. No.: US 2020/0063208 A1
(43) Pub. Date: Feb. 27, 2020

(54) EXOSOMAL BIOMARKERS FOR DIAGNOSIS AND PROGNOSIS OF CANCER AND RELATED METHODS

(71) Applicant: University of Louisville Research Foundation, Inc., Louisville, KY (US)

(72) Inventor: Huang-Ge Zhang, Louisville (US)

(73) Assignee: University of Louisville Research Foundation, Inc., Louisville, KY (US)

(21) Appl. No.: 16/340,457

(22) PCT Filed: Oct. 13, 2017
(86) PCT No.: PCT/US17/56570

§ 371 Date: Apr. 9, 2019

Related U.S. Application Data
(60) Provisional application No. 62/407,690, filed on Oct. 13, 2016.

(51) Int. Cl. C12Q 1/6886; C12Q 1/686

(52) U.S. Cl. CPC C12Q 1/6886; C12Q 1/686; C12Q 2600/158; C12Q 2600/118; C12Q 2600/178

(57) ABSTRACT

Methods for diagnosing a cancer in a subject are provided in which a biological sample is provided from a subject including one or more exosomes. The exosomes are isolated from the biological sample and are used to identify a type and/or amount of one or more tumor suppressor miRNAs encapsulated by the exosomes. A measurable difference in the type and/or amount of the tumor suppressor miRNAs as compared to control levels allows for a diagnosis of cancer in the subject. Methods of selecting or modifying a treatment and methods for determining whether to initiate or continue prophylaxis or treatment for a cancer based on the exosomal miRNAs is further provided.

### RELATED APPLICATIONS

This application claims priority from U.S. Provisional Application Ser. No. 62/407,690, filed Oct. 13, 2016, the entire disclosure of which is incorporated herein by this reference.

### GOVERNMENT INTEREST

This invention was made with government support under grant numbers UH3TR000875 and R01AT008617 awarded by the National Institutes of Health. The government has certain rights in the invention.

### TECHNICAL FIELD

The presently-disclosed subject matter generally relates to exosomal biomarkers for diagnosis and prognosis of cancer. In particular, certain embodiments of the presently-disclosed subject matter relate to methods for diagnosing cancer in a subject based on the identification of one or more tumor suppressor miRNAs present in the exosomes of a subject.

### BACKGROUND

Extracellular vesicles (EVs) or exosomes have emerged as important mediators of intercellular communication in a diverse range of biological processes and are being utilized as biomarkers for disease diagnosis and prognosis. Although it is appreciated that cancer cells secrete EVs that promote cancer progression and that these EVs contain markers for potential cancer diagnosis and prognosis, a complete working model of EV mediated biological effects has not been demonstrated in a fully physiological in vivo context. A fully working model is urgently needed in the EV field for understanding the in vivo fate of cancer EVs which could have implications for developing therapeutic strategies against cancer. Most studies published thus far analyze the function of EV populations isolated from the supernatants of cultured cells. One of the challenges is whether EV secretion in vitro by tumor cells is able to achieve this function in vivo is still not clear. A number of approaches have been undertaken to address this question by either interfering in in vivo EV biogenesis in cancer cells with siRNA knockdown or chemical inhibitors to inhibit EV releasing. However, the specificity of this effect for EV secretion, even when considering what has been reported with cell culture derived EVs, has not been warranted. Moreover, EVs including exosomes are released from many different types of cells including cancer cells as well as non-cancer cells. Yet, without a known cancer exosome specific marker, the biological effect of cancer cell derived exosomes versus non-cancer cell derived exosomes cannot be defined.

### SUMMARY

The presently-disclosed subject matter meets some or all of the above-identified needs, as will become evident to those of ordinary skill in the art after a study of information provided in this document.

This summary describes several embodiments of the presently-disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter includes exosomal biomarkers for diagnosis and prognosis of cancer. In particular, the presently-disclosed subject matter includes methods for diagnosing a cancer in a subject based on the identification of one or more tumor suppressor miRNAs present in the exosomes of a subject. In some embodiments of the presently-disclosed subject matter, a method for diagnosing a cancer in a subject is provided that comprises the steps of: providing a biological sample from the subject; isolating exosomes from the biological sample; identifying an amount of one or more tumor suppressor miRNAs in the exosomes; and comparing the amount of the one or more tumor suppressor miRNAs in the exosomes, if present, to a control level of the one or more tumor suppressor miRNAs. Based on the measurable difference in the amounts of the one or more tumor suppressor miRNAs encapsulated by the exosomes, the subject can then be diagnosed as having cancer or a risk thereof. In some embodiments, a treatment for the cancer is selected or modified based on the identification of the one or more tumor suppressor miRNAs in the exosomes.

In some embodiments of the presently-described methods for diagnosing a cancer, the cancer is selected from the group consisting of colon cancer and liver cancer. In some embodiments, the cancer is a primary cancer or a secondary cancer. In some embodiments, the subject has cancer. In some embodiments, the subject is a human.

With respect to the biological sample that is provided, in some embodiments, the biological sample comprises, for example, blood, plasma, serum, or feces as each of those biological samples has been found to be a source of exosomes. In some embodiments, to identify one or more tumor suppressor miRNAs in the exosomes isolated from the biological sample, the identification is performed using polymerase chain reaction (PCR) or microarray analysis and/or comprises providing a probe for selectively binding each of the one or more miRNAs. In some embodiments, the one or more tumor suppressor miRNAs comprise one or more miRNAs selected from the miRNAs of Table 2. In some embodiments, the miRNA is miR-193a.

Further provided in some embodiments of the presently-disclosed subject matter are methods for determining whether to initiate or continue prophylaxis or treatment of a cancer in a subject. In some embodiments, a method for determining whether to initiate or continue prophylaxis or treatment of a cancer in a subject is provided that comprises the steps of: providing a series of biological samples over a time period from the subject; isolating exosomes from each biological sample in the series of biological samples; identifying an amount of one or more tumor suppressor miRNAs in the exosomes isolated from each biological sample of the series of biological samples; and comparing any measurable change in the amounts of the one or more tumor suppressor miRNAs in the exosomes in each biological sample of the series of biological samples. Based on the measurable change identified, a determination is then made as to whether to initiate or continue the prophylaxis or therapy of the cancer. In some embodiments, the series of biological samples comprises a first biological sample collected prior to initiation of the prophylaxis or treatment for cancer and a second biological sample collected after initiation of the prophylaxis or treatment. In other embodiments, the series of biological samples comprises a first biological sample collected prior to onset of the cancer and a second biological sample collected after the onset of the cancer.

Further features and advantages of the presently-disclosed subject matter will become evident to those of ordinary skill in the art after a study of the description, figures, and non-limiting examples in this document.

### BRIEF DESCRIPTION OF THE DRAWINGS

FIGS. 1A-1H include graphs and schematic diagrams showing the identification of exosome miRNA profiles that represent primary colon cancer and liver metastasis of colon cancer, including: (FIG. 1A) a schematic diagram for isolation of extracellular vesicles (EVs) from colon cancer CT26 cell line with multimodal imaging report, where CT26 cells were stably transduced with a lentiviral vector expressing membrane-bound Gaussia luciferase (GlucB) and biotin ligase (BirA); (FIG. 1B) a venn diagram summarizing unique and shared exosomal miRNAs detected in the tissues of naïve colon, primary colon cancer, and liver metastasis of mouse colon cancer using miRNA microarray data (n=5 mice per group); (FIG. 1C) a microarray data visualization by scatter plot comparing exosomal miRNAs detected in primary colon cancer (X-axis) and liver metastasis of colon cancer at day 3 (Y-axis) after CT26 intra-splenic injection; (FIG. 1D) a heat map depicting changes in miRNAs with a statistically significant (p<0.05) change in the exosomal miRNAs from normal mouse colon, primary colon cancer tissue, and liver metastasis of colon cancer at day 3, 7 and 14 after injection of CT26 (n=3 mice per group), where all tumor derived exosomes were isolated with streptavidin magnetic beads; (FIG. 1E) microarray analysis results and (FIG. 1F) qPCR verification of selected exosomal miRNAs from the source as described in the FIG. 1D; (FIG. 1G) qPCR analysis of the plasma (top panel) or feces (bottom panel)-derived exosomes from the source as descripted in the FIG. 1D; and (FIG. 1H) a network prediction by Ingenuity Pathway Analysis (IPA) for higher and lower levels of exosomal miRNAs from liver metastasis of colon tumor, where pointed arrowheads represent relationships, while solid or dash line indicates direct or indirect relationships, respectively. In FIGS. 1A-1G, *P<0.05 versus naïve colon; #, p<0.05 versus primary colon cancer (two-tailed t-test). Data are representative of three independent experiments (error bars, S.E.M.).

FIGS. 2A-2D includes graphs showing that the miRNA profile from exosomes is different from their donor cells; including (FIG. 2A) a graph showing a comparative analysis of miRNome in exosomes and exosome donor tissues using microarray, where miRNAs from exosomes and exosome donor tissues including primary colon cancer (top panel) and liver metastasis (middle panel) were quantitatively analyzed and expressed as a ratio of miRNAs from exosome donor tissues/exosomes, and where the similarity of each individual miRNA distribution in primary colon cancer and liver metastasis was analyzed by overlaying each and are shown in a yellow color (bottom panel); and graphs showing the expression of miR-193a (FIG. 2B), miR-18a (FIG. 2C) and miR-21 (FIG. 2D) in the exosomes and exosome donor tissues, including primary colon cancer and liver metastasis of colon cancer, assessed by qPCR. In FIGS. 2A-2D, *P<0.05 (two-tailed t-test). Data are representative of three independent experiments (error bars, S.E.M.).

FIG. 3 includes graphs showing that microenvironment alters composition of tumor exosomes miRNAs profiles where exosomal miRNAs isolated from the sources indicated in FIG. 3 were quantitatively analyzed by qPCR, where *P<0.05 (two-tailed t-test), and where data are representative of three independent experiments (error bars, S.E.M.).

FIGS. 4A-4H include schematic diagrams, graphs, and images showing that miR-193a suppresses the progression of CT26 colon cancer by directly targeting Caprin1, including: (FIG. 4A) a schematic diagram of the putative binding sites of miR-193a (SEQ ID NO: 14) in the wild type (WT) Caprin1 3′ untranslated regions (UTR), where the miR-193a seed matches in the Caprin1 3′UTR are mutated at the positions as indicated (SEQ ID NO: 13). CDS, coding sequence; (FIG. 4B) a schematic diagram showing conserved sites of Caprin1 potentially to be bound by miR-193a (in gray) broadly conserved among vertebrates (Mouse: (SEQ ID NO: 15); Rabbit (SEQ ID NO: 15); Human (SEQ ID NO: 15); Chimpanzee (SEQ ID NO: 15); Dog (SEQ ID NO: 15); Cat (SEQ ID NO: 15); Chicken (SEQ ID NO: 16); and Frog (SEQ ID NO: 17); (FIG. 4C) a graph showing expression of miR-193a and candidate target genes Caprin1 as well as downstream genes (Ccnd1, c-myc) in CT26 cells assessed by qPCR following transfection of miR-193a mimic and control scramble miRNA; (FIG. 4D) images showing expression of candidate miR-193a and candidate target genes Caprin1 as well as downstream genes (CCND2, c-myc, G3BP1) in CT26 cells assessed by western blot, following transfection of miR-193a mimic and control miRNA for 72 h; (FIG. 4E) a graph showing proliferation of CT26 cells with miR-193a and potential target Caprin1 knock down, where cell viability was detected from day 0 to 5 after transfection; (FIG. 4F) a graph showing luciferase activity assays of wild type (WT) and mutated Caprin1 3′UTR luciferase reporters after co-transfection with miR-193a mimic, miRNA mimic control (scramble), anti-sense miR-193a, or anti-sense negative control RNA in CT26 cells, where the luciferase activity of each sample was normalized to the Renilla luciferase activity, and where the normalized luciferase activity of transfected control mimic miRNA was set as relative luciferase activity of 1; (FIG. 4G) a graph showing survival of BALB/c mice after intrasplenic injection of CT26 cells with miR-193 overexpression (n=6 mice per group); and (FIG. 4H) a graph showing the cell cycle phase analysis of CT26 cells transfected with miR-193a mimic for 72 h, where the percent values of cells in the G1, S, G2 phase are shown in the bar graph. In FIGS. 4A-4G, error bars represent S.E.M. *P<0.05 and **P<0.01, and each data point was measured in triplicate (error bars, S.E.M.).

FIGS. 5A-5I include images and graphs showing sorting of miR-193a from cell to exosomes through Major vault protein (MVP), including: (FIG. 5A) an image and graph showing a biotin-miR-193a complex pulled down from whole cell extracts using streptavidin beads and then analyzed by electrophoresis followed by Coomassie blue staining (left panel) as well as MALDI-TOF analysis of tryptic peptides (right panel) from the band indicated (left panel); (FIG. 5B) images showing western blot analysis expression of MVP proteins from before (top panel) and after streptavidin pulldown (bottom panel) of lysates of CT26 cells transfected with Bio-miR-193a or control miRNA; (FIG. 5C) a graph showing the results of MVP knockout (KO) CT26 cells generated using the CRISPR/Cas9 system followed by qPCR-quantification of mature miR-193a, MVP, Caprin1, CyclinD and c-myc expressed in CT26 cells (left panel) and CT26 exosomes (right panel) after the cells were treated as indicated; (FIG. 5D) images showing western blot analysis of the level of MVP, Caprin1, CCND2 and c-myc in cell lysates treated as indicated; (FIG. 5E) a graph showing the proliferation of MVP KO CT26 cells treated as indicated, where cell viability was detected from day 0 to 5 after transfection; (FIG. 5F) a schematic representation (top panel, first page) of treatment schedule as indicated, images of representative livers with metastatic nodules shown by arrows and H&E-stained sections of livers (bottom panel, first page) at 400× magnification from tumor bearing BALB/c mice (n=5 per group), and graphs showings liver weight (top panel, second page) and number of metastasis foci in liver (second page) quantitatively analyzed; (FIG. 5G) graphs showing mature miR-193a in tumor tissue and tumor exosomes quantified by qPCR along with a survival analysis of BALB/c mice after intra-splenic injection of CT26 cells treated as indicated (n=9 per group); (FIG. 5H) representative images of xenografts in SW620 tumor bearing nude mice (n=5 mice per group) and graphs showing changes of tumor volumes in a SW620 xenograft model, where the volume of liver tumor was used to evaluate tumor size using the following formula: nodules volume=(width)2×length/2; and (FIG. 5I) graphs showing qPCR-quantification of mature miR-193a in exosomes and tissues of tumor in SW620 xenograft mice. In FIGS. 5A-5I, *P<0.05 and each data point was measured in triplicate (error bars, S.E.M.).

FIGS. 6A-6E include graphs, images, and schematic diagrams showing that induction of exosome miR-193a in peripheral blood increases the risk of liver metastasis in colon cancer patients, including: (FIG. 6A) graphs showing qPCR-quantification of mature miR-193a, miR-126a, miR-148a and miR-196b in exosomes of plasma collected from colon cancer patient with (n=15) or without (n=25) liver metastasis; (FIG. 6B) a graph showing qPCR analysis of miR-193a level in the exosomes from peripheral blood of colon cancer patients without metastasis and follow up investigation carried out six month after diagnosis; (FIG. 6C) representative H&E-stained sections of colon tumor tissue (200× magnification) from patients in various stages and graphs showing qPCR analysis of miR-193a, miR-126a and miR-148a expression in colon cancer tissue and adjacent non-tumor tissue from the same patients; (FIG. 6D) images showing representative MVP (red) expression in patient tumor sections with Alexa Fluor 594 dye labeling anti-MVP antibodies (red), visualized with a confocal microscopy; (FIG. 6E) images showing western blot analysis of the level of MVP, Caprin1, Cyclin D and c-myc in colon cancer tissue (C) and adjacent non-tumor tissue (A) from the same patient in various stages (I, II, and III) as indicated, where GAPDH was used as a loading control. In FIGS. 6A-6E, data are representative of three independent experiments, and *P<0.05 and **P<0.01 (error bars, S.E.M.).

FIG. 7 is a schematic diagram showing a proposed model for the mechanism of colon cancer liver metastasis involves miR-193a exporting through exosomes sorted by MVP (abbreviations: MVB, multivesicular bodies; RISC, RNA-induced silencing complex).

FIGS. 8A-8B include images and a graph showing the characterization of stable expression of GlucB and sshBirA in the CT26 cell line, including (FIG. 8A) images showing live cell imaging of stable CT26 cells expressing green fluorescent protein (GFP) conjugated membrane-bound Gaussia luciferase (GlucB) with or without biotin ligase (BirA); and (FIG. 8B) graphs showing activity of a reporter using Gluc luciferase assay with Gluc substrate coelentrazine (CTZ) in the exosomes from the stable CT26 cell line, medium and cell lysates (RLU; relative light units). In FIGS. 8A-8B, data are representative of three independent experiments, and *P<0.05 (error bars, s.e.m.).

FIGS. 9A-9C include images showing an in vivo expression of GlucB and sshBirA in the stable CT26 cells; including: (FIG. 9A) images showing representative endoscopic views of mouse colon and hematoxylin and eosin (H&E)-stained sections of colon tissue, where 1×10⁶ stable EV-GlucB expressing CT26 cells were administered to BALB/c mice via colon submucosal or intra-splenic injection, and where representative colon, liver, and representative H&E-stained sections of formalin-fixed, paraffin-embedded colon and liver (400× magnification) from naive BALB/c and tumor bearing mice at 14 d after administration of CT26 cells are shown; (FIG. 9B) images showing visualization of CT26 cell stably expressing GlucB-GFP and BirA in the sectioned liver by confocal microscopy, where metastases liver of CT26 was collected from BALB/c mice 2 weeks after intrasplenic injection of 1×106 CT26 cells; and (FIG. 9C) images showing in vivo imaging of EV-GlucB expression in CT26 cells, where BALB/c mice were administered vehicle (control) or CTZ by intravenous injection 2 weeks after intra-splenic injection of 1×106 stable CT26 cells.

FIGS. 10A-10E are images showing purification and characterization of exosomes from liver metastasis of colon cancer using an EV-GlucB reporter, including: (FIG. 10A) images showing dot blot detection of EV-GlucB using exosomal marker CD63 antibody and streptavidin, where exosomes were isolated from naïve liver and liver metastatic CT26 cell line which was stably expressing GlucB and BirA; (FIG. 10B) images showing expression of CD63 and endoplasmic reticulum protein Calnexin in exosomes and tissue from liver metastasis of colon cancer assessed by western blot, following transfection of miR-193a mimic and control miRNA for 72 h; (FIG. 10C) representative electron microscopic images of exosomes from liver metastasis of colon cancer and naïve liver; (FIG. 10D) images showing size distribution of exosomes from naïve liver or liver metastasis of colon cancer analyzed using a Zetasizer Nano ZS; and (FIG. 10E) a graph showing quantification of size distribution of exosomes derived from naïve liver and liver metastasis of colon cancer. In FIGS. 10A-10E, *P<0.05 (two-tailed t-test), and data are representative of three independent experiments (error bars, S.E.M.)

FIGS. 11A-11B are graphs showing microarray data visualization by scatter plot and comparing exosome miRNAs (X-axis) with donor tissue miRNAs (Y-axis) in colon cancer (FIG. 11A) and naïve colon (FIG. 11B) (Differential expression of log 2 value >2), where, in the scatter plot, each point represents the expression value of a gene, the green and red dots represent the genes highly expressed in exosomes and tissues, respectively, and the grey dots represent similar gene expression between exosomes and tissues.

FIG. 12 is a graph showing specific induction of miR-193a in MVP KO cells, where MVP KO cells were generated using the CRSPR/Cas9 system, where selected miRNAs, as well as MVP expression, in cell lysates were evaluated by qPCR, and where *P <0.05 and **P<0.01 (two-tailed t-test) and data are representative of three independent experiments (error bars, S.E.M.).

FIG. 13 is a graph showing that higher levels of exosomal miR193a in mouse feces associated with increased numbers of colon tumors, and that drinking curcumin provides protection against colon development.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

Sequences described herein are described with reference to GENBANK® accession numbers and SWISSPROT identification numbers. The sequences cross-referenced in the GENBANK® and SWISSPROT databases are expressly incorporated by reference as are equivalent and related sequences present in GENBANK®, SWISSPROT, or other public databases. Also expressly incorporated herein by reference are all annotations present in the GENBANK®, and SWISSPROT databases associated with the sequences disclosed herein. Unless otherwise indicated or apparent the references to the GENBANK® database and the SWISSPROT database are references to the most recent version of the database as of the filing date of this Application.

In certain instances, microRNAs (miRNAs) disclosed herein are identified with reference to names assigned by the miRBase Registry (available at www.mirbase.org). The sequences and other information regarding the identified miRNAs as set forth in the miRBase Registry are expressly incorporated by reference as are equivalent and related miRNAs present in the miRBase Registry or other public databases. Also expressly incorporated herein by reference are all annotations present in the miRBase Registry associated with the miRNAs disclosed herein. Unless otherwise indicated or apparent, the references to the Sanger miRBase Registry are references to the most recent version of the database as of the filing date of this Application (i.e., mirBase 21, released June 2014). See, e.g., miRNA Accession numbers provided in Table 2 below.

The present application can “comprise” (open ended), “consist of,” or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

The terms “polypeptide”, “protein”, and “peptide”, which are used interchangeably herein, refer to a polymer of the 20 protein amino acids, including modified amino acids (e.g., phosphorylated, glycated, etc.) and amino acid analogs, regardless of size or function. Although “protein” is often used in reference to relatively large polypeptides, and “peptide” is often used in reference to small polypeptides, usage of these terms in the art overlaps and varies. The term “peptide” as used herein refers to peptides, polypeptides, proteins and fragments of proteins, unless otherwise noted. The terms “protein”, “polypeptide” and “peptide” are used interchangeably herein when referring to a gene product and fragments thereof. Thus, exemplary polypeptides include gene products, naturally occurring proteins, homologs, orthologs, paralogs, fragments and other equivalents, variants, fragments, and analogs of the foregoing.

The terms “polypeptide fragment” or “fragment”, when used in reference to a polypeptide, refers to a polypeptide in which amino acid residues are absent as compared to the full-length polypeptide itself, but where the remaining amino acid sequence is usually identical to the corresponding positions in the reference polypeptide. Such deletions can occur at the amino-terminus or carboxy-terminus of the reference polypeptide, or alternatively both. A fragment can retain one or more of the biological activities of the reference polypeptide. In some embodiments, a fragment can comprise a domain or feature, and optionally additional amino acids on one or both sides of the domain or feature, which additional amino acids can number from 5, 10, 15, 20, 30, 40, 50, or up to 100 or more residues. Further, fragments can include a sub-fragment of a specific region, which sub-fragment retains a function of the region from which it is derived. When the term “peptide” is used herein, it is intended to include the full-length peptide as well as fragments of the peptide. Thus, an identified fragment of a peptide (e.g., by mass spectrometry) is intended to encompass the fragment as well as the full-length peptide. As such, determining an amount of a biomarker in a sample can include determining an amount of the full-length biomarker polypeptide, modified variants, and/or fragments thereof.

The presently-disclosed subject matter is based, at least in part, on the discovery that tumor exosomes selectively sort tumor suppressor miRNA into exosomes, while oncogenic miRNA is kept in the tumor cell regardless of the level of miRNA expressed in the cell. The presently-disclosed subject matter thus provides methods, assays, and systems for diagnosis and prognosis of cancer, including the progression of a cancer. In some embodiments, the presently-disclosed subject matter includes methods and systems for diagnosing cancer in a subject, and for determining whether to initiate or continue prophylaxis or treatment of cancer in a subject, by identifying an amount of one or more tumor suppressor miRNAs in exosomes isolated from a biological sample from a subject. In some embodiments, the one or more tumor suppressor miRNAs are the selected from the miRNAs of Table 2. In some embodiments, the one or more tumor suppressor miRNAs comprise miR-193a.

MicroRNAs are naturally occurring, small non-coding RNAs that are about 17 to about 25 nucleotide bases (nt) in length in their biologically active form. miRNAs post-transcriptionally regulate gene expression by repressing target mRNA translation. It is thought that miRNAs function as negative regulators, i.e. greater amounts of a specific miRNA will correlate with lower levels of target gene expression. There are three forms of miRNAs existing in vivo, primary miRNAs (pri-miRNAs), premature miRNAs (pre-miRNAs), and mature miRNAs. Primary miRNAs (pri-miRNAs) are expressed as stem-loop structured transcripts of about a few hundred bases to over 1 kb. The pri-miRNA transcripts are cleaved in the nucleus by an RNase II endonuclease called Drosha that cleaves both strands of the stem near the base of the stem loop. Drosha cleaves the RNA duplex with staggered cuts, leaving a 5′ phosphate and 2 nt overhang at the 3′ end. The cleavage product, the premature miRNA (pre-miRNA) is about 60 to about 110 nt long with a hairpin structure formed in a fold-back manner. Pre-miRNA is transported from the nucleus to the cytoplasm by Ran-GTP and Exportin-5. Pre-miRNAs are processed further in the cytoplasm by another RNase II endonuclease called Dicer. Dicer recognizes the 5′ phosphate and 3′ overhang, and cleaves the loop off at the stem-loop junction to form miRNA duplexes. The miRNA duplex binds to the RNA-induced silencing complex (RISC), where the antisense strand is preferentially degraded and the sense strand mature miRNA directs RISC to its target site. It is the mature miRNA that is the biologically active form of the miRNA and is about 17 to about 25 nt in length. In this regard, the phrase “tumor suppressor miRNAs” is used herein to refer to mature miRNAs whose function is to reduce the growth or progression or development of tumor cells in a subject, such as by, for example, inhibiting oncogene expression. Conversely, the phrase “tumor miRNAs” or “oncogenic miRNAs” is used herein to refer to mature miRNAs that function as a class of molecules or oncogenes capable of promoting tumor development, such as by negatively inhibiting tumor suppressor genes.

The exemplary human biomarkers disclosed are not intended to limit the present subject matter to human miRNA biomarkers only. Rather, the present subject matter encompasses tumor suppressor miRNAs across animal species that are associated with cancer. A “biomarker” is a molecule useful as an indicator of a biologic state in a subject. With reference to the present subject matter, the biomarkers disclosed herein can be miRNAs that exhibit a change in expression or state, such as being found in (i.e., encapsulated by exosomes), which can then be correlated with the risk of developing, the presence of, or the progression of cancer in a subject.

In some embodiments of the presently-disclosed subject matter, a method for diagnosing cancer. in a subject is provided. The terms “diagnosing” and “diagnosis” as used herein refer to methods by which the skilled artisan can estimate and even determine whether or not a subject is suffering from a given disease or condition. The skilled artisan often makes a diagnosis on the basis of one or more diagnostic indicators, such as for example a marker, the amount (including presence or absence) of which is indicative of the presence, severity, or absence of the condition.

Along with diagnosis, clinical disease prognosis is also an area of great concern and interest. It is important to know the stage and rapidity of advancement of the cancer in order to plan the most effective therapy. If a more accurate prognosis can be made, appropriate therapy, and in some instances less severe therapy for the subject can be chosen. Measurement of biomarker levels disclosed herein (e.g., the miRNAs listed in Table 2) can be useful in order to categorize subjects according to advancement of cancer who will benefit from particular therapies and differentiate from other subjects where alternative or additional therapies can be more appropriate.

As such, “making a diagnosis” or “diagnosing”, as used herein, is further inclusive of determining a prognosis, which can provide for predicting a clinical outcome (with or without medical treatment), selecting an appropriate treatment (or whether treatment would be effective), or monitoring a current treatment and potentially changing the treatment, based on the measure of diagnostic biomarker levels disclosed herein.

The phrase “determining a prognosis” as used herein refers to methods by which the skilled artisan can predict the course or outcome of a condition in a subject. The term “prognosis” does not refer to the ability to predict the course or outcome of a condition with 100% accuracy, or even that a given course or outcome is predictably more or less likely to occur based on the presence, absence or levels of test biomarkers. Instead, the skilled artisan will understand that the term “prognosis” refers to an increased probability that a certain course or outcome will occur; that is, that a course or outcome is more likely to occur in a subject exhibiting a given condition, when compared to those individuals not exhibiting the condition. For example, in individuals not exhibiting the condition (e.g., not expressing the biomarker or expressing it at a reduced level), the chance of a given outcome may be about 3%. In certain embodiments, a prognosis is about a 5% chance of a given outcome, about a 7% chance, about a 10% chance, about a 12% chance, about a 15% chance, about a 20% chance, about a 25% chance, about a 30% chance, about a 40% chance, about a 50% chance, about a 60% chance, about a 75% chance, about a 90% chance, or about a 95% chance.

The skilled artisan will understand that associating a prognostic indicator with a predisposition to an adverse outcome is a statistical analysis. For example, a biomarker level (e.g., quantity of expression in a sample) of greater than a control level in some embodiments can signal that a subject is more likely to suffer from a cancer than subjects with a level less than or equal to the control level, as determined by a level of statistical significance. Additionally, a change in marker concentration from baseline levels can be reflective of subject prognosis, and the degree of change in marker level can be related to the severity of adverse events. Statistical significance is often determined by comparing two or more populations, and determining a confidence interval and/or a p value. See, e.g., Dowdy and Wearden, Statistics for Research, John Wiley & Sons, New York, 1983, incorporated herein by reference in its entirety. Preferred confidence intervals of the present subject matter are 90%, 95%, 97.5%, 98%, 99%, 99.5%, 99.9% and 99.99%, while preferred p values are 0.1, 0.05, 0.025, 0.02, 0.01, 0.005, 0.001, and 0.0001.

In other embodiments, a threshold degree of change in the level of a prognostic or diagnostic biomarker can be established, and the degree of change in the level of the indicator in a biological sample can simply be compared to the threshold degree of change in the level. A preferred threshold change in the level for markers of the presently-disclosed subject matter is about 5%, about 10%, about 15%, about 20%, about 25%, about 30%, about 50%, about 75%, about 100%, and about 150%. In yet other embodiments, a “nomogram” can be established, by which a level of a prognostic or diagnostic indicator can be directly related to an associated disposition towards a given outcome. The skilled artisan is acquainted with the use of such nomograms to relate two numeric values with the understanding that the uncertainty in this measurement is the same as the uncertainty in the marker concentration because individual sample measurements are referenced, not population averages.

In some embodiments of the presently-disclosed subject matter, multiple determination of one or more diagnostic or prognostic peptide biomarkers can be made, and a temporal change in the biomarker can be used to monitor the progression of disease and/or efficacy of appropriate therapies directed against the disease. In such an embodiment for example, one might expect to see a decrease or an increase in the biomarker(s) over time during the course of effective therapy. Thus, the presently disclosed subject matter provides in some embodiments a method for determining treatment efficacy and/or progression of a cancer in a subject. In some embodiments, the method comprises determining an amount of one or more miRNAs encapsulated by exosomes isolated from biological samples from a subject, such as for example at least one miRNA in Table 2, in biological samples collected from the subject at a plurality of different time points and comparing the amounts of the miRNAs in the samples collected at different time points. For example, a first time point can be selected prior to initiation of a treatment and a second time point can be selected at some time after initiation of the treatment. One or more biomarker levels can be measured in each of the samples taken from different time points and qualitative and/or quantitative differences noted. A change in the amounts of the biomarker levels from the first and second samples can be correlated with determining treatment efficacy and/or progression of the disease in the subject.

The terms “correlated” and “correlating,” as used herein in reference to the use of diagnostic and prognostic biomarkers, refers to comparing the presence or quantity of the biomarker in a subject to its presence or quantity in subjects known to suffer from, or known to be at risk of, a given condition (e.g., a cancer); or in subjects known to be free of a given condition, i.e. “normal individuals.” For example, a miRNA profile (e.g., type of miRNAs or levels of miRNAs) in exosomes isolated from a biological sample can be compared to a level known to be associated with a specific type of cancer. The sample's biomarker level is said to have been correlated with a diagnosis; that is, the skilled artisan can use the biomarker level to determine whether the subject suffers from a specific type or stage of advancement of cancer, and respond accordingly. Alternatively, the sample's biomarker profile (i.e., type and/or level) can be compared to a control marker level known to be associated with a good outcome (e.g., the absence of cancer), such as an average level found in a population of normal subjects.

In certain embodiments, a diagnostic or prognostic biomarker is correlated to a condition or disease by merely its presence or absence. In other embodiments, a threshold level of a diagnostic or prognostic biomarker can be established, and the level of the indicator in a subject sample can simply be compared to the threshold level.

As noted, in some embodiments, multiple determination of one or more diagnostic or prognostic biomarkers can be made, and a temporal change in the marker can be used to determine a diagnosis or prognosis. For example, a diagnostic marker can be determined at an initial time, and again at a second time. In such embodiments, an increase in the marker from the initial time to the second time can be diagnostic of a particular type or progression of cancer, or a given prognosis. Likewise, a decrease in the marker from the initial time to the second time can be indicative of a particular type or progression of cancer, or a given prognosis. Furthermore, the degree of change of one or more markers can be related to the severity of the cancer and future adverse events.

The skilled artisan will understand that, while in certain embodiments comparative measurements can be made of the same diagnostic marker at multiple time points, one can also measure a given marker at one time point, and a second marker at a second time point, and a comparison of these markers can provide diagnostic information.

In some embodiments, a method for diagnosing a cancer in a subject is provided that comprises the steps of: providing a biological sample from the subject; isolating exosomes from the biological sample; identifying an amount of one or more tumor suppressor miRNAs in the exosomes; and comparing the amount of the one or more tumor suppressor miRNAs in the exosomes, if present, to a control level of the one or more tumor suppressor miRNAs. Based on the measurable difference in the amounts of the one or more tumor suppressor miRNAs encapsulated by the exosomes in the biological sample, the subject can then be diagnosed as having cancer or a particular type or stage of cancer, or a risk thereof.

With regard to the step of providing a biological sample from the subject, the term “biological sample” as used herein refers to any body fluid or tissue potentially comprising exosomes. In some embodiments, for example, the biological sample can be a blood sample, a serum sample, a plasma sample, or sub-fractions thereof. In some embodiments, the biological sample can be feces.

Turning now to the step of identifying one or more markers in the biological sample, various methods known to those skilled in the art can be used to identify the one or more markers in the provided biological sample. In some embodiments, determining the amount of biomarkers in samples comprises using a miRNA measuring assay to measure miRNA in an exosome obtained from a biological sample from a subject. For example, in some embodiments of the presently-disclosed subject matter, exosomes are isolated and purified from a biological sample using centrifugation techniques and the one or more miRNAs in the sample are identified by making use of one or more probes that selectively bind the miRNA molecules present or suspected of being present in the exosomes. In this regard, the term “probe” is used herein to refer to a biopolymer comprising one or more nucleic acids, nucleotides, nucleosides and/or their analogs. The term also includes nucleotides having modified sugars as well as organic and inorganic leaving groups attached to the purine or pyrimidine rings, where each probe has a binding affinity for a different miRNA sequence. In some embodiments, the term “probe” is thus further inclusive of the term “primer,” which can be used in a variety of methods of identifying an miRNA in an exosome isolated from a subject, including, for example, polymerase chain reaction (PCR), reverse-transcriptase (RT)-PCR, RNA PCR, LCR, multiplex PCR, panhandle PCR, capture PCR, expression PCR, 3′ and 5′ RACE, in situ PCR, ligation-mediated PCR and other amplification protocols. In some embodiments, identifying the one or more tumor suppressor miRNAs in the isolated exosomes comprises identifying the miRNAs using polymerase chain reaction and/or microarray analysis. Of course, the identification and development of probes specific for an miRNA of interested can be performed using methods well known to those skilled in the art.

In some embodiments of the presently-disclosed subject matter, the tumor suppressor miRNAs described herein can be identified alone or in combination with one or more polypeptides (e.g., Major Vault Protein, MVP) associated with the sorting of those miRNAs into exosomes. As such, and with regard to determining amounts of biomarker miRNAs in samples, mass spectrometry and/or immunoassay devices and methods can be used to measure or identify biomarker miRNAs in samples through the measurement or identification of certain peptides known to be associated with a the presence or absence of a particular miRNA in an exosome.

With further respect to the measurement and detection of the miRNAs in a biological sample, although certain embodiments of the method only call for a qualitative assessment of the presence or absence of the one or more markers in the biological sample, other embodiments of the method call for a quantitative assessment of the amount of each of the one or more markers in the biological sample. Such quantitative assessments can be made, for example, using one of the above mentioned methods, as will be understood by those skilled in the art.

In certain embodiments of the methods described herein, it can also be desirable to include a control sample that is analyzed concurrently with the biological sample, such that the results obtained from the biological sample can be compared to the results obtained from the control sample. Additionally, it is contemplated that standard curves can be provided, with which assay results for the biological sample can be compared. Such standard curves present levels of biomarkers as a function of assay units, i.e., fluorescent signal intensity, if a fluorescent signal is used. Using samples taken from multiple donors, standard curves can be provided for control levels of the one or more markers in normal tissue.

Additionally, in some embodiments of the presently-disclosed subject matter, it is contemplated that the efficacy, accuracy, sensitivity, and/or specificity of the method can be enhanced by probing for multiple markers (e.g., multiple tumor suppressor miRNAs) in exosomes in the biological sample. The analysis of markers can be carried out separately or simultaneously with additional markers within one test sample. For example, several markers can be combined into one test for efficient processing of a multiple samples and for potentially providing greater diagnostic and/or prognostic accuracy. In addition, one skilled in the art would recognize the value of testing multiple samples (for example, at successive time points) from the same subject. Such testing of serial samples can allow the identification of changes in marker levels over time. Increases or decreases in marker levels, as well as the absence of change in marker levels, can provide useful information about the disease status that includes, but is not limited to identifying the approximate time from onset of the cancer, the presence and amount of salvageable tissue, the appropriateness of drug therapies, the effectiveness of various therapies, and identification of the subject's outcome, including risk of future events.

Clinical cancer prognosis is also an area of great concern and interest. It is important to know the aggressiveness of the cancer cells and/or the likelihood of tumor recurrence in order to plan the most effective therapy. If a more accurate prognosis can be made, appropriate therapy, and in some instances less severe therapy for the subject can be chosen. In some embodiments of the presently-disclosed subject matter, the identification of exosomes in a sample obtained from a subject can be used to determine the progression of the cancer in the subject and/or whether the cancer is a primary cancer or a secondary cancer.

As used herein, the term “primary cancer” is meant to refer to an original tumor or cancer cell in a subject. Such primary cancers are usually named for the part of the body in which the primary cancer originates. Furthermore, a “secondary cancer” is used herein to refer to a cancer which has spread, or metastasized, from an initial site (i.e. a primary cancer site) to another site in the body of a subject, a cancer which represents a residual primary cancer, or a cancer that has originated from treatment with an antineoplastic agent(s) or radiation or both. In this regard, the term “secondary cancer” is thus not limited to any one particular type of cancer, including the type of primary cancer from which it derived. In some embodiments of the presently-disclosed subject matter, a method of preventing or treating a cancer is further provided where the subject is at risk of developing a secondary cancer.

As used herein, the term “cancer” refers to all types of cancer or neoplasm or malignant tumors found in animals, including leukemias, carcinomas, melanoma, and sarcomas. By “leukemia” is meant broadly progressive, malignant diseases of the blood-forming organs and is generally characterized by a distorted proliferation and development of leukocytes and their precursors in the blood and bone marrow. Leukemia diseases include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

The term “carcinoma” refers to a malignant new growth made up of epithelial cells tending to infiltrate the surrounding tissues and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tubero sum, tuberous carcinoma, verrucous carcinoma, and carcinoma villosum.

The term “sarcoma” generally refers to a tumor which is made up of a substance like the embryonic connective tissue and is generally composed of closely packed cells embedded in a fibrillar or homogeneous substance. Sarcomas include, for example, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilns' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumor arising from the melanocytic system of the skin and other organs. Melanomas include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma subungal melanoma, and superficial spreading melanoma.

Additional cancers include, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, and adrenal cortical cancer. In some embodiments, the cancer is selected from colon cancer and liver cancer.

With respect to the subjects of the presently-disclosed subject matter, a preferred subject is a vertebrate subject. A preferred vertebrate is warm-blooded; a preferred warm-blooded vertebrate is a mammal. A preferred mammal is most preferably a human. As used herein, the term “subject” includes both human and animal subjects. Thus, veterinary therapeutic uses are provided in accordance with the presently-disclosed subject matter. As such, the presently-disclosed subject matter provides for the diagnosis of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

The practice of the presently disclosed subject matter can employ, unless otherwise indicated, conventional techniques of cell biology, cell culture, molecular biology, transgenic biology, microbiology, recombinant DNA, and immunology, which are within the skill of the art. Such techniques are explained fully in the literature. See e.g., Molecular Cloning A Laboratory Manual (1989), 2nd Ed., ed. by Sambrook, Fritsch and Maniatis, eds., Cold Spring Harbor Laboratory Press, Chapters 16 and 17; U.S. Pat. No. 4,683,195; DNA Cloning, Volumes I and II, Glover, ed., 1985; Oligonucleotide Synthesis, M. J. Gait, ed., 1984; Nucleic Acid Hybridization, D. Hames & S. J. Higgins, eds., 1984; Transcription and Translation, B. D. Hames & S. J. Higgins, eds., 1984; Culture Of Animal Cells, R. I. Freshney, Alan R. Liss, Inc., 1987; Immobilized Cells And Enzymes, IRL Press, 1986; Perbal (1984), A Practical Guide To Molecular Cloning; See Methods In Enzymology (Academic Press, Inc., N.Y.); Gene Transfer Vectors For Mammalian Cells, J. H. Miller and M. P. Calos, eds., Cold Spring Harbor Laboratory, 1987; Methods In Enzymology, Vols. 154 and 155, Wu et al., eds., Academic Press Inc., N Y; Immunochemical Methods In Cell And Molecular Biology (Mayer and Walker, eds., Academic Press, London, 1987; Handbook Of Experimental Immunology, Volumes I-IV, D. M. Weir and C. C. Blackwell, eds., 1986.

The presently-disclosed subject matter is further illustrated by the following specific but non-limiting examples.

### EXAMPLES

Materials and Methods for Examples 1-7

Isolation and Purification of Exosomes.

To isolate exosomes from liver tissue, a 20-G catheter was inserted into the portal vein of anaesthetized mice. The inferior vena cava was cut to allow the pre-warmed (37° C.). perfusion buffer (Ca²⁺—Mg²⁺ free HBSS containing 0.5 mM EGTA, 10 mM HEPES and 4.2 mM NaHCO₃; pH 7.2) to flow freely through the liver. The liver was perfused 7-10 ml/min until there was no evidence of blood in the spent perfusion medium. The liver was then perfused for 5 min with dissociation buffer (HBSS containing 10 mM HEPES and 4.2 mM NaHCO₃ supplemented with Type I collagenase (0.05%) and trypsin inhibitor (50 μg/ml); pH 7.5) pre-warmed at 37° C. The perfused liver and xenograft from subcutaneous and submucosa colon cancer tissue, as well as naïve colon tissue were removed and gently disaggregated with tweezers in dissociation buffer, followed by incubation at 37° C. for 1 h. The disaggregated samples were centrifuged at 1,000 g for 10 min, 2,000 g for 20 min, 4,000 g for 30 min and 10,000 g for 1 h with supernatant being retained each time. The tissue exosomes were collected by centrifuging the samples at 100,000 g for 1.5 h at 4° C. and the pellet suspended in ice-cold PBS or used for RNA isolation immediately. To isolate exosomes from FBS exosomes-free cell culture medium, the cell supernatants were collected the exosomes purified by differential centrifugation using a previously described method. The exosomes were further purified on a sucrose gradient (8, 30, 45 and 60% sucrose in 20 mM HEPES, 20 mM Tris-Cl, pH 7.2).

Exosomes from serum and feces were isolated using the exoEasy Maxi Kit (Qiagen, Frederick, Md.) according to the manufacturer's instructions. Briefly, 1 volume Buffer XBP was added to 1 volume of pre-filtered serum or feces suspended in PBS using Millex-AA (Millipore, Billerica, Mass.) and then mixed well. The sample/Buffer XBP mix was then added onto the exoEasy spin column. After washing with 10 ml Buffer XWP, the exosomes were eluted using 400 μl Buffer XE. The exosomes were further purified on a sucrose gradient (8, 30, 45 and 60% sucrose in 20 mM HEPES, 20 mM Tris-Cl, pH 7.2). Size distribution of exosomes was analyzed using a Zetasizer Nano ZS (Malvern Instrument, UK).

Isolation of Biotinylated Exosomes.

Isolated exosomes were resuspended in 90 μl of labeling buffer (2 mM EDTA/PBS) (per 1 mg exosomes) and incubated with 10 μl of streptavidin MicroBeads (Miltenyi Biotec, Germany) in 500 μl of labeling buffer at 4° C. for 2 h. After three washes with separation buffer (2 mM EDTA and 0.5% BSA/PBS), biotinylated exosomes were ready for protein extraction or RNA isolation.

Generation of stable CT26 cell line expressing EV-GlucB and sshBirA.

The stable HEK293T packaged cells expressing Gluc or GlucB with sshBirA were generated by transduction with lentivirus expression plasmids. All plasmids were transfected with lentivirus packing vectors pCMVdelta8.2 and VSV-G using the FuGENE reagent (Promega, WI). Pseudovirus-containing culture medium was collected after 72 h of transfection and the viral titer estimated. 2×10⁵ CT26 cells in a 6-well plate received 10 μg/mL of polybrene as well as appropriate amount of viral stock in medium. After selection by puromycin, the cells with the highest expression of EV-GlucB and sshBirA were sorted using a BD FACSAria™ III cell sorter (BD Biosciences, San Jose, Calif.).

Clinical Samples.

All clinical samples including tissue and serum samples were collected with written informed consent was obtained from patients in the Department of Surgery, Huai'an First People's Hospital, Huai'an, Jiangsu, China. Approval for the study was granted by the Institute Research Ethics Committee at the Health Department of Huai'an. Stages of colon cancer reflect whether and how far the cancer had spread according to the criteria from the National Cancer Institute.

Cell Culture.

The BALB/c syngeneic colon carcinoma CT26 cell line, human colonic epithelial SW620 cell line, and human embryonic kidney 293 cells (American Type Culture Collection, Rockville, Md.) were grown at 37° C. in 5% CO₂ in Dulbecco's Modified Eagle's medium (DMEM, Life technology) supplemented with 10% heat-inactivated EV-depleted fetal bovine serum (FBS), 100 U/mL penicillin, and 100 μg/mL streptomycin. To isolate exosomes from cell culture medium, samples were centrifuged at 120,000 g for 12 h at 4° C.

Mouse Model Study.

8- to 12-week-old female BALB/c mice and athymic immunodeficient nude mice were purchased from the Jackson Laboratory (Bar Harbor, Me.) and housed under specific pathogen-free conditions. Animal care was performed following the Institute for Laboratory Animal Research (ILAR) guidelines and all animal experiments were done in accordance with protocols approved by the University of Louisville Institutional Animal Care and Use Committee (Louisville, Ky.). The mice were acclimated for at least 1 week before any experiments were conducted.

For animal models of liver metastasis of colon cancer, mice were anaesthetized with a mixture of ketamine and xylazine by intraperitoneal injection and 1×10⁶ CT26 colon cancer cells were administered via intra-splenic injection as previously described. For the animal xenograft model, female nude mice were inoculated subcutaneously on both flanks with human colonic epithelial SW620 cells (1×10⁶). On day 14, mice were sacrificed and livers or tumors were removed for examination. Xenograft volumes were evaluated by caliper measurements of two perpendicular diameters and calculated individually as formula: Volume=a×b²/2 (a represents length and b represents width). Xenograft samples were collected for engraftment, histologic evaluation (paraffin section) or exosome isolation. The blood samples were obtained by cardiac puncture and fractionated by centrifugation and the plasma was stored at −80° C. until ready for use.

For an animal model of primary colon cancer, 1×10⁵ CT26 colon cancer cells were injected into the colonic submucosa of BALB/c mice (n=5) via an endoscopic work station (Karl Storz-Endoskope, Tuttlingen, German). The mice were sacrificed and colon tumors were removed for study on day 14-21 post-injection.

Quantitative Real-Time PCR (qPCR) Analysis of miRNA and mRNA Expression.

Total RNA was isolated from cells, exosomes and tissue using a miRNeasy mini kit (Qiagen), from serum using the exoRNeasy Serum/Plasma Midi Kit (Qiagen). For the isolation of RNA from paraffin-embedded tissues, 4 sections at 5 microns were placed in a tube, deparaffinized using xylene (Fisher) and rehydrate by decreasing concentrations of ethanol and PBS. Total RNA was isolated with the miRNeasy mini kit (Qiagen). The quantity of mature miRNAs expressed was determined by quantitative real-time PCR (qPCR) using a miScript II RT kit (Qiagen) and miScript SYBR Green PCR Kit (Qiagen) with Qiagen predesigned primers. All kits were used according to the manufacturer's instructions. A U6 transcript was used as an internal control to normalize RNA input. For analysis of MVP, Caprin1, c-myc, cyclin D1 mRNA expression, 1 μg of total RNA was reverse transcribed by SuperScript III reverse transcriptase (Invitrogen) and quantitation was performed using primers (Eurofins) with SsoAdvanced™ Universal SYBR Green Supermix (BioRad). GAPDH was used for normalization. The primer sequences are listed in Table 1 below. qPCR was run using the BioRad CFX96 qPCR System with each reaction run in triplicate. Analysis and fold-change were determined using the comparative threshold cycle (Ct) method. The change in miRNA or mRNA expression was calculated as fold-change.

TABLE 1
Primer sequences used for quantitative
Real-Time PCR (qPCR) of mRNA

| | Primers | Forward (5′-3′) | Reverse (5′-3′) |
|---|---|---|---|
| qPCR | MVP | AGACGAGTGGCTGTTTGAG | CAGAGCTTGGTTCTGTT |
| | | (SEQ ID NO: 1) | TGATG (SEQ ID NO: 2) |
| | CAPRIN1 | CTTATGGCACAAATGCAAG | CATGTTCTGGGTAGGGT |
| | | GG (SEQ ID NO: 3) | TCATAG (SEQ ID NO: 4) |
| | CCND2 | GAAGGACATCCAACCGTAC | TTCATGGCCAGAGGAAA |
| | | AT (SEQ ID NO: 5) | GAC (SEQ ID NO: 6) |
| | C-MYC | GCGATCAGCTCTCCTGAAA | GCAGAAAGAACACAGG |
| | | (SEQ ID NO: 7) | GAAAG (SEQ ID NO: 8) |
| | GAPDH | GGTCGGTGTGAACGGATTT | GGAGTCATACTGGAACA |
| | | G (SEQ ID NO: 9) | TGTAG (SEQ ID NO: 10) |
| Mutant | Mutantgenesis | GTGTTTTTGGCGATTAAAC | ACTGACTCGTGTACCAT |
| | | ATAATCCTG | AATATGTTACCAG |
| | | (SEQ ID NO: 11) | (SEQ ID NO: 12) |

Analysis of miRNA Microarray.

miRNA expression profiling was performed using the Qiagen miScript miRNA PCR Array Mouse miRBase Profiler (Cat #331223). Quantile normalization and subsequent data processing were performed using software R. Heat maps and scatter plots representing differentially regulated genes were generated using software R. Ingenuity pathway analysis (IPA) was used to generate a network for predicting target genes in exosomes with high or low levels of miRNAs in liver metastasis of colon cancer.

Site-Directed Mutagenesis within the Caprin1 Promoter.

Two online algorithms that predict the mRNA targets of miRNAs were utilized, TargetScan and microRNA, and Pictar. Caprin1 was selected by both online tools with strong conserved 3′ untranslated region (3′ UTR) sites. To determine the ability of miR-193a to target the 3′UTR-Caprin1 activity, a luciferase reporter containing 3,243 bp of the Caprin1 3′UTR in the pEZX-MT01 vector was purchased from GeneCopoeia (Cat^{#}: MmiT072744-MT06, Rockville, Md.). The mutant of Caprin1 3′UTR was generated with the oligonucleotide primer Caprin1-Mut, which was designed to specifically disrupt putative Caprin1 at its 3′ UTR site. Q5® Site-Directed Mutagenesis Kit (New England Biolabs, MA, USA) was used in conjunction with specific primers (Table 1) to introduce Caprin1 3′ UTR mutations in the pEZX-MT01 construct according to the manufacturer's instructions. After mutant strand synthesis and ligation, resultant plasmids were introduced into E. coli and transformants were selected using kanamycin resistance. The DNA sequence of mutants was confirmed by DNA sequencing.

Transient Transfection and Luciferase Reporter Assay.

Murine colon cancer CT26 cells were plated in 24-well plates at a density of 3.0×10⁴ cells/well in antibiotic free RPMI-1640 medium supplemented with 10% FBS. 100 ng of Caprin1-pEZX-MT01 or mutant luciferase reporter were transfected using FuGENE HD Transfection Reagent (Roche Applied Science, Indianapolis, Ind.) with 10 pmol of mimic mmu-miR-193a and Opti-MEM® Reduced Serum Medium (Invitrogen, Carlsbad, Calif.). For all reporter assays, the cells were harvested 48 h post-transfection using Promega's Passive Lysis buffer. The activities of luciferase in cell lysates were determined using the Dual-Luciferase Reporter Assay System (Promega). Relative expression (fold-change) was determined by dividing the averaged normalized values from mock transfection. Values were averaged as indicated in the Figure legends.

Protein Identification by Proteomic Analysis by MALDI-TOF Mass Spectrometry.

Protein bands were excised from 10% SDS-PAGE gel stained with Colloidal coomassie blue (Bio-Rad, Hercules, Calif.) and incubated in 50 mmol/L NH₄HCO₃/50% acetonitrile at 22° C. for 15 min The gel pieces were allowed to swell by incubating with 20 mmol/L DTT in 0.1 mol/L NH₄HCO₃ for 45 min at 56° C. After removing the DTT solution, the gel was incubated in 55 mmol/L iodoacetamide in 0.1 mol/L NH₄HCO₃ for 30 min in the dark. The gel was rinsed with 50 mmol/L NH₄HCO₃ and incubated in 50 mmol/L NH₄HCO₃/50% acetonitrile. After drying in a speedvac, an aliquot of 25 μg/mL sequencing-grade trypsin in 50 mmol/L NH₄HCO₃ was added. After a 45-min incubation on ice, the supernatant was discarded and replaced with 20 μL of 50 mmol/L NH₄HCO₃. Digestion was done at 37° C. overnight and fragmented peptides were extracted from the gel with 5% formic acid/50% acetonitrile. To improve the ionization efficiency of matrix-assisted laser desorption/ionization time-of-flight mass spectrometry (MALDI-TOF-MS), ZipTipC18 (Millipore) was used to purify peptides before MS analysis, according to the manufacturer's manual. The peptides were eluted with 2 μL of 5 mg/mL α-cyano-4-hydroxycinnamic acid in 50% acetonitrile/0.1% Trifluoroacetic acid and applied directly onto the plate and allowed to air dry. Peptide mass fingerprints were obtained using a TOF-Spec 2E MALDI-TOF mass spectrophotometer (Waters). The Mascot program (Matrix Science) was used to interpret MS spectra of protein digests.

Western Blotting.

Cells were treated as indicated in individual Figure legends and whole cell extracts (WCE) were prepared in modified radioimmunoprecipitation assay (RIPA) buffer (Sigma) with addition of protease and phosphatase inhibitors (Roche). Western blot analysis was performed and quantitated as described previously. Proteins were separated by 10% SDS-PAGE and transferred to PVDF membranes (Bio-Rad Laboratories, Inc., Hercules, Calif.). Dual color precision protein MW markers (BioRad) were separated in parallel. Antibodies were purchased as follows: MVP (Cat #: 16478-1-AP) from ProteinTech, CD63 (Cat #: 143902) from Biolegend, Calnexin (Cat #: C45520) from Transduct. Lab, Caprin1 (Cat #: sc-83115), c-myc (Cat #: sc-41), cyclinD2 (Cat #: sc-181) and α-tubulin (Cat #: sc-8035) from Santa Cruz Biotechnology (Santa Cruz, Calif.). The secondary antibodies conjugated to Fluors Alex-647 were purchased from Invitrogen (Eugene, Oreg.). The bands were visualized and analyzed on an Odyssey Imager (LiCor Inc, Lincoln, Nebr.).

Histological Analysis.

Tissues were fixed with buffered 10% formalin solution (SF93-20; Fisher Scientific, Fair Lawn, N.J.) overnight at 4° C. Dehydration was achieved by immersion in a graded ethanol series, i.e., 70%, 80%, 95%, 100% ethanol for 40 min each. Tissues were embedded in paraffin and subsequently cut into ultra-thin slices (5 μm) using a microtome. Deparaffinization was accomplished using xylene (Fisher) and rehydration using decreasing concentrations of ethanol and PBS. Tissue sections were stained with hematoxylin and eosin (H&E) and slides were scanned with an Aperio ScanScope. For tissue immunofluorescent staining, slides were washed three times (5 minutes each) with PBST (PBS, 0.1% Tween 20). The tissue was permeabilized by incubating the slides in 1% Triton X-100 in PBS at 25° C. for 15 minutes and then washed three times in PBST. After blocking for 1 h at 25° C. in blocking buffer (PBS containing 10% bovine serum albumin (BSA)), slides were incubated overnight in a humidity chamber with anti-MVP polyclonal antibody (ProteinTech). Antibodies were diluted 1:50 in blocking buffer. Following another three PBST washes, slides were incubated with Alexa 647-conjugated secondary antibody at a 1:500 dilution (Invitrogen). Slides were then washed and nuclei counterstained with 4′,6-Diamidino-2-phenylindole dihydrochloride (DAPI).

For frozen sections, tissues were fixed with periodate-lysine-paraformaldehyde (PLP) and dehydrated with 30% sucrose in PBS at 4° C. overnight. Tissue sections were stained with primary Ab in PBS/5% BSA (1:200) for 2 h and secondary Ab in PBS/5% BSA (1:800) for 30 min DAPI was used for nuclear stain.

Gaussia luciferase (Gluc) Activity Assays.

To test Gluc activity of CT26 stably expressing EV-GlucB, 1×10⁶ CT26 cells were grown in exosome-free culture medium for 72 h. 1 μg of exosomes from medium following treatment with RIPA lysis buffer (Sigma), 1 μg of whole cell lysates or 100 μl of medium were used for the Gluc activity assay. The activities of luciferase were determined using the BioTek's Synergy Microplate Reader after incubation with 50 μl of Coelenterazine (CTZ) (10 ng/mL, Nanolight). Values were averaged as indicated in the Figure legends.

Imaging of EV-GlucB Distribution In Vivo.

To evaluate EV-GlucB bioluminescence activity in tumor-bearing mice, five BALB/c mice received by intrasplenic injection CT26 cells stably expressing EV-GlucB. On day 14 after tumor cell inoculation, mice were injected with 50 μl of CTZ (50 ng/mL, Nanolight) or PBS as control and bioluminescence activity was imaged 5 min after the injection using an Advanced Molecular Imager AMI (Spectral Instruments Imaging, AZ) connected to an anesthesia system (Summit, UT).

Electron Microscopy of Isolated Exosomes.

Isolated exosomes in PBS were fixed in 2% paraformaldehyde (Electron Microscopy Science, PA) in PBS for 2 h at 22° C. followed by 1% glutaraldehyde (Electron Microscopy Science, PA) for 30 min at 22° C. 15 μl of fixed samples were put on 2% agarose with formvar/carbon-coated nickel grids on top and allowed to absorb for 5-10 min. The grids with adherent exosomes were fixed in 2% paraformaldehyde in PBS for 10 min followed by extensive washing in PBS. Negative contrast staining was performed with 1.9% methyl cellulose and 0.3% uranyl acetate for 10 min The grids with negatively stained exosomes were dried before observation under a Zeiss EM 900 electron microscope.

Dot Blots Analysis of Biotinylated Exosomes.

Exosome from GlucB stable expressive CT26 liver metastasis purified with streptavidin beads and exosomes from naïve liver were lysed with RIPA buffer. 1 μg of lysate was spotted onto nitrocellulose membranes and blocked with 5% BSA in PBS at 4° C. overnight. CD63 and biotin were visualized by Alexa Fluor® (Fisher) fluorescent conjugated anti-CD63 antibody and streptavidin at 4° C. overnight respectively.

RNA Interference.

CT26 cells were grown to 70% confluency in six-well plates in antibiotic-free DMEM supplemented with 5% FBS. Cells were transfected with 90 pmoles miRNA or siRNA/well using 7 μl of RNAiMAX (Invitrogen) in antibiotic-free medium and incubated for 48-72 h. As a control, cells were transfected with scramble control miRNA or siRNA (Ambion). RNA and protein lysates were prepared for qPCR and Western blot analysis.

Flow Cytometric Cell Cycle Analysis.

Analysis of cell cycle distribution was carried out by flow cytometric analysis of propidium iodide (PI)-stained cells. 1×10⁶ cells were fixed with 70% ethanol for 1 h at −20° C. The samples were then centrifuged at 1000 g for 5 min. The 70% ethanol was removed, and the cells were then treated with 100 μl of RNase A (0.5 mg/ml) for 30 min at 37° C. Cell samples were then stained with 20 μg/ml of propidium iodide and analyzed with a BD FACS Calibur flow cytometer to obtain DNA content profiles. FlowJo was used for analysis of cell cycle.

Cell Proliferation Assay.

Cell proliferation assays were performed using the Cell Titer96 AQueous One Solution Cell Proliferation Assay from Promega. Briefly, 1×10³ CT26 cells were plated per well in 96-well plates. Cells were treated with miRNA or siRNA indicated in the Figure legends for 48 h to 5 d depending on the experiment. Absorbance was measured at 490 nm using a 96-well plate reader SpectraMax M2 (Molecular Devices, Sunnyvale, Calif.). Each treatment was performed in quadruplicate within each experiment.

Statistical Analysis.

One-way analysis of variance (ANOVA) followed by Turkey Post Hoc tests was used to determine the differences occurring between more than two groups, and T test was used to determine the difference between two groups (*p<0.05, **p<0.01). All statistical analyses in this study were performed with SPSS 16.0 software. Data are presented as mean±SD. The significance of mean values between two groups was analyzed by Student's t test. Differences between individual groups were analyzed by one- or two-way analysis of variance test. Differences were considered significantly when the P value was less than 0.05 or 0.01 as indicated in the text.

### Example 1—Isolation and Characterization of Tumor Specific Exosomes

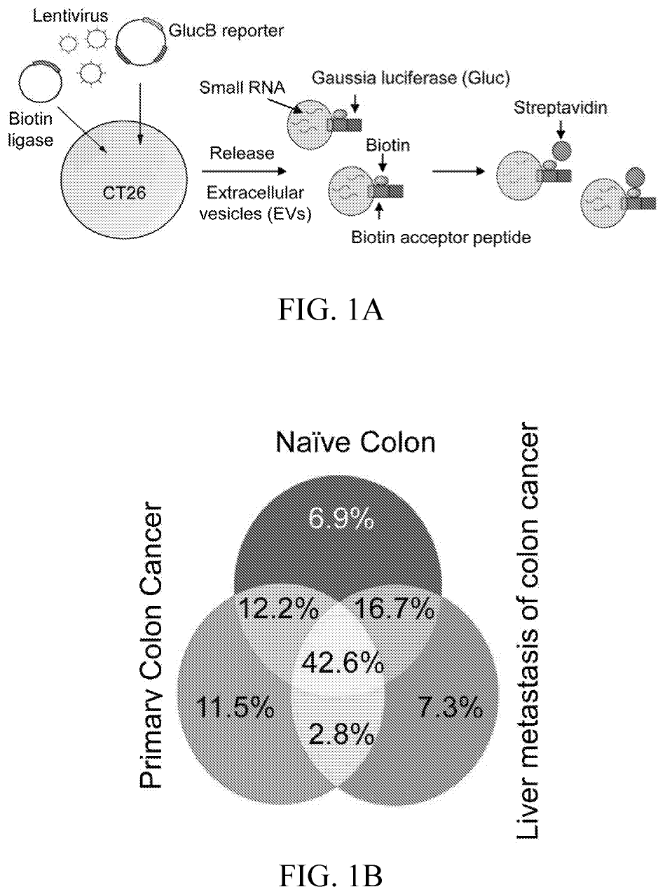

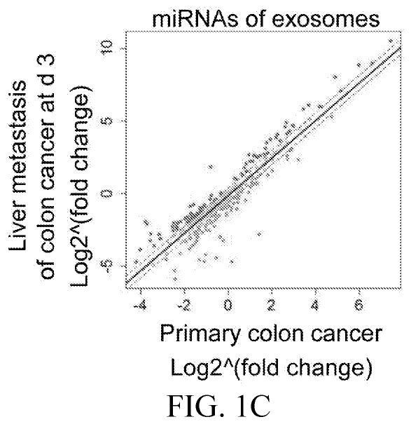

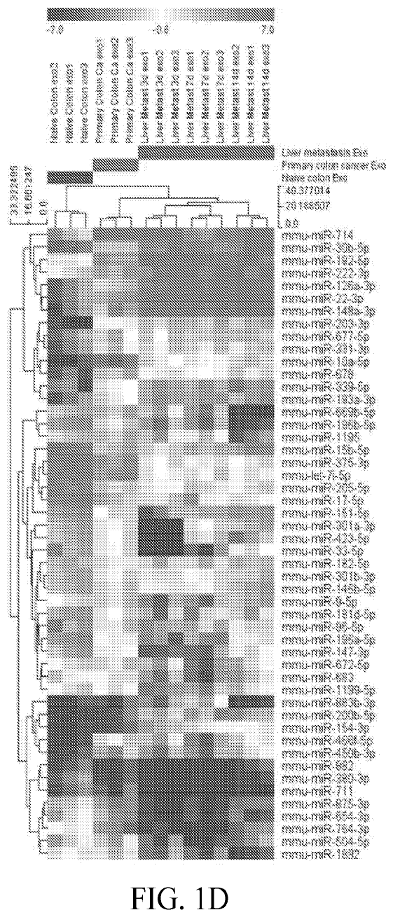

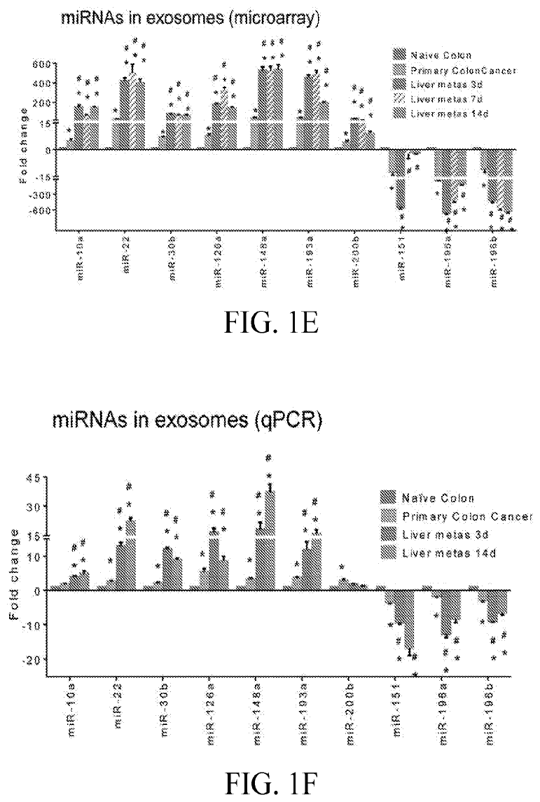

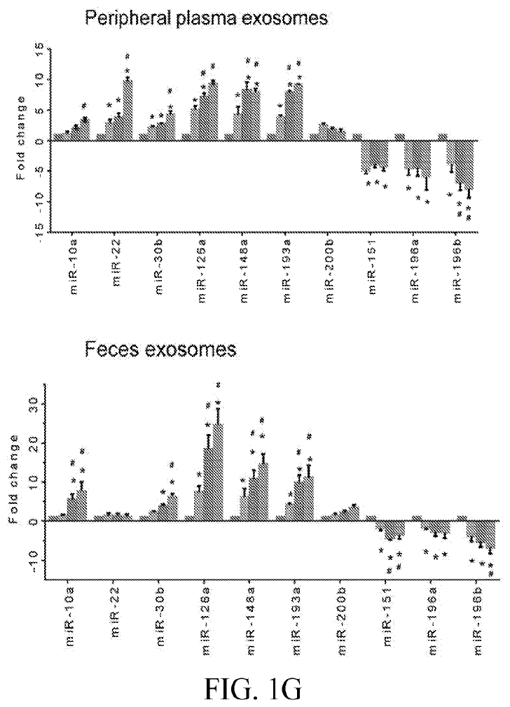

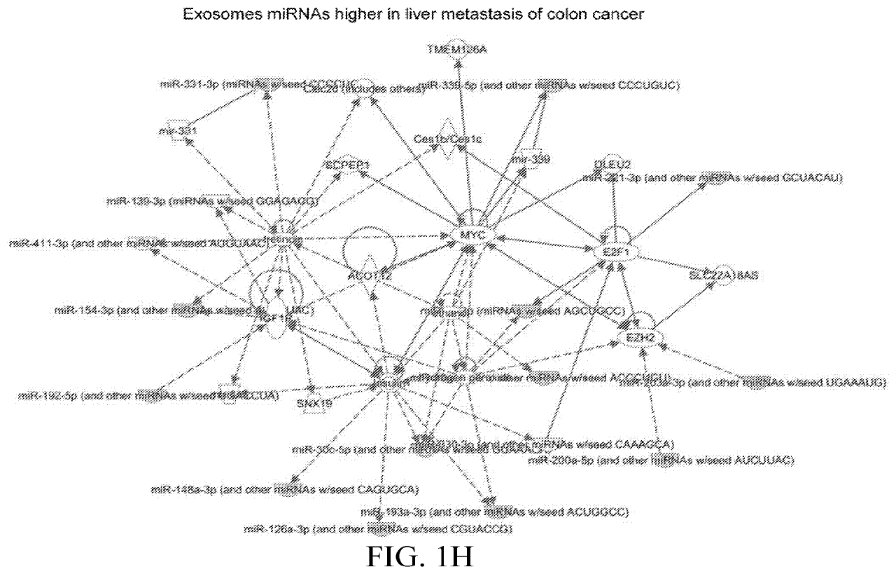

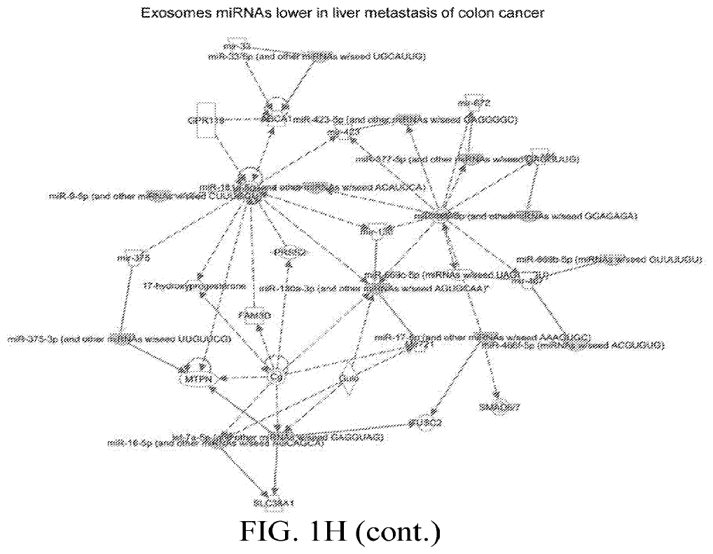

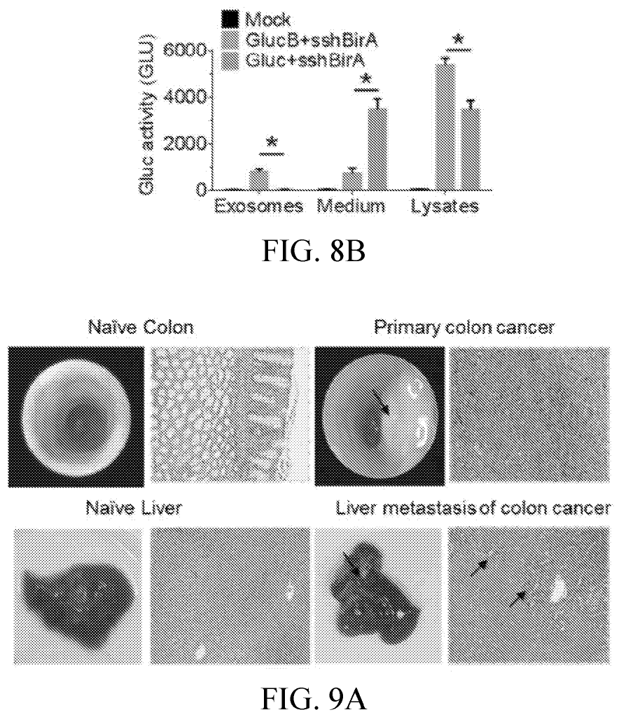

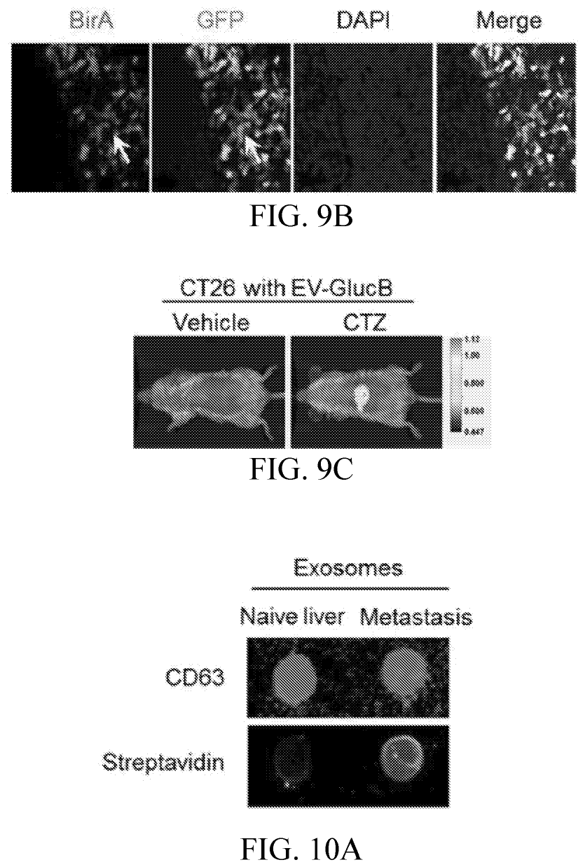

The discovery of Extracellular Vesicle (EV) RNA presence not only in tissues but also in fluids, including blood, together with their changes in expression in various pathological conditions, has implicated EV RNAs as informative biomarkers of progression and early diagnosis for cancer. However, EVs are released from many different types of cells including tumor and non-tumor cells. The challenge is to distinguish between EVs released from tumor cells and the EVs released from non-tumor cells. To achieve this overall goal, stably transduced colon cancer CT26 cells were generated with lentivirus vectors which enabled the isolation of EVs from CT26 tumor cells (FIG. 1A). The expression of this construct provided a biotin binding moiety for isolation of exosomes, as well as Gaussia luciferase (Gluc) and green fluorescent protein (GFP) fluorescent tags for monitoring EV levels in organs and biological fluids (FIG. 8A). The results generated from luciferase assays with Coelenterazine (CTZ) indicate that the EVs released from CT26 cells stably transfected with the lentivirus vector expressing Gluc, biotin acceptor peptide (BAP), and transmembrane (TM) domain exhibited a higher luciferase activity compared with the cells expressing Gluc without the TM domain (FIG. 8B). Moreover, more Gluc activity in the medium and less Gluc activity in the whole cell lysates has been detected in the cells transfected with membrane-bound Gluc than GlucB. Next, to characterize the tumor EVs released from primary and metastatic cancer in the liver of colon cancer mice, CT26 cells stably expressed both EV-Gluc and biotin ligase BirA were administered to BALB/c mice by colonic submucosa or intra-splenic injection as previously described. Two weeks after injection, tumor was evident in hematoxylin and eosin (H&E) stained sectioned colon and liver (FIG. 9A) and confocal imaging of sectioned liver (FIG. 9B). EV-Gluc and BirA expression were visualized with GFP-tagged Gluc and red fluorescent mCherry-tagged BirA in metastatic lesions in the liver, but not in the adjacent normal liver tissue (FIG. 9B). To estimate EV-Gluc luciferase activity in vivo, CTZ or phosphate-buffered saline (PBS; control) was injected by tail vein injection into BALB/c mice bearing Gluc expression CT26 tumor. Five minutes after the injection, CTZ injected mice revealed a significant amount of Gluc signal in the liver of EV-GlucB expressing mice, but not PBS injected tumor bearing mice (FIG. 9C). These results confirm an in vivo biological activity and stability of EV-Gluc imaging reporter in tumor cells.

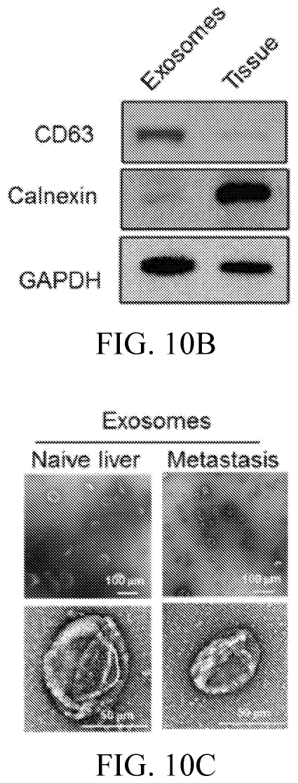

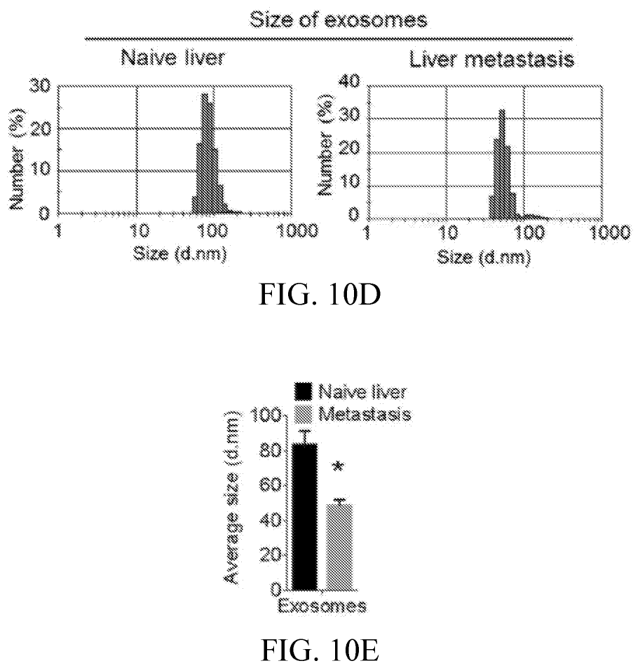

To further determine if the protocol used was capable of isolating tumor specific exosomes, exosomes were isolated from metastatic liver of colon cancer followed by purification with streptavidin beads to eliminate potential contamination with other extracellular microvesicles. Isolated exosomes were dot blotted on nitrocellulose membrane followed by probing with anti-CD63 antibody and streptavidin conjugated Alexa Fluor 488. Dot blot analysis showed that exosomes from both naïve mouse liver and liver with metastatic colon cancer expressed exosomal marker CD63 with the same fluorescent intensity (FIG. 10A). However biotinylated EV-GlucB could be detected by streptavidin, but not naïve mouse liver-derived exosomes (FIG. 10A). Western blot demonstrated the presence of exosomal protein marker CD63 and the absence of the endoplasmic reticulum protein Calnexin from exosomes (FIG. 10B). Exosomal morphology and size distribution were further evaluated using electron microscopy (FIG. 10C) and Zetasizer Nano ZS analysis (FIG. 10D), respectively. Liver metastatic exosomes had a diameter of 48.6±4.6 nm (means±standard error of the mean (SEM)) and were smaller than naïve mice liver-derived exosomes which had a diameter of 83.6±7.8 nm (FIG. 10E).

### Example 2—Tumor Exosomal miRNA as an Indicator for Tumor Progression

Identification of a unique miRNA profile encapsulated in tumor cell exosomes but not in non-tumor cell exosomes is required for any clinical application. Tumor exosomal miRNA has recently been extensively studied for use as a diagnostic marker and also as an indicator for disease progression. To further determine whether the presently-described approach can identify an exosomal miRNA profile that mirrors disease progression, a miRNA microarray and comparative analysis of the miRNome was performed in exosomes isolated from normal colon, primary colon cancer tissue, and colon tumor metastasis to the liver (FIG. 1B, FIG. 9A). Among these differentially expressed miRNAs, 6.9% of the miRNAs was uniquely detected in normal colon tissue derived exosomes; 11.5% of the miRNAs was detected in primary colon tumor exosomes; and 7.3% of the miRNAs was detected in metastasis tumor exosomes (FIG. 1B). Next, exosomal miRNA profiles from metastatic liver of colon cancer were compared with the exosomal miRNA profiles from primary colon tumor. In the scatter plot (FIG. 1C) each point represents the expression value of a given miRNA. Compared with exosomal RNAs from primary colon tumor, exosomal RNA profiles from metastatic liver of colon cancer displayed a different distribution (FIG. 1C). The green and red dots represent the higher level of exosomal miRNAs detected in the primary colon cancer and liver metastasis, respectively, and the grey dots represent similar levels of exosomal miRNAs detected in primary colon cancer and liver metastasis. The criteria used to screen differences in miRNA encapsulation in the exosomes between normal tissue versus primary colon tumor, and primary colon tumor versus metastatic tumor were based on the fold-changes of >3.0 or <−3.0. Fifty of the miRNAs met these criteria and were selected for further analysis (Table 2). A heat map of the fifty selected miRNAs demonstrated gene cluster and sample cluster according to the level of miRNAs in the exosomes from normal tissue, primary colon tumor and metastatic liver of colon cancer (FIG. 1D). To verify the microarray results, seven of the up-regulated miRNAs and three of down-regulated miRNAs in metastatic liver of colon cancer (FIG. 1E) were randomly chosen and confirmed by qPCR (FIG. 1F). The qPCR results indicated that these miRNAs were actually encapsulated into exosomes and subsequently released into the circulation (FIG. 1G, left panel) and excreted into the gut (FIG. 1G, right panel) from liver metastatic CT26 tumor cell. The stable character of exosomal miRNAs in biological fluids and feces indicates that miRNAs in exosome can be used as potential biomarkers for clinical diagnosis and prognosis. Using Ingenuity Pathway Analysis (IPA) to identify the networks and pathways, the pathways regulated by miRNAs that are either over-(FIG. 1H) or under-(FIG. 1H) expressed in the CT26 colon tumor exosomes from metastatic liver were analyzed. The network generated using IPA revealed the interactions of miRNAs enriched in the exosomes from metastatic liver of colon cancer with potential target genes such as IGF1R, EZH2, E2F1 and MYC that have oncogenic function. In contrast, miRNAs in limited or lower amounts in the exosomes from metastatic liver of colon cancer interact with genes such as SMAD6/7, TUSC2, MTPN and ABCA1 that involve tumor suppressor function.

TABLE 2
List of significant higher level or lower level miRNAs presented in the tumor exosomes (fold change).

| | Accession | Primary cancer/ | Metast/Primary | | Accession | Primary cancer/ | Metast/Primary |
|---|---|---|---|---|---|---|---|
| Higher in tumor exo | number | Naive (log2) | cancer (log2) | Lower in tumor exo | number | Naive (log2) | cancer (log2) |
| mmu-miR-10a-5p | MIMAT0000648 | 2.17 | 5.22 | mmu-miR-423-5p | MIMAT0004825 | −2.44 | −10.21 |
| mmu-miR-126a-3p | MIMAT0000138 | 6.21 | 4.76 | mmu-miR-301a-3p | MIMAT0000379 | −3.11 | −10.04 |
| mmu-miR-22-3p | MIMAT0000531 | 8.01 | 4.19 | mmu-miR-33-5p | MIMAT0000667 | −5.73 | −7.22 |
| mmu-miR-192-5p | MIMAT0000517 | 4.11 | 4.11 | mmu-miR-9-5p | MIMAT0000142 | −1.93 | −5.92 |
| mmu-miR-339-5p | MIMAT0000584 | 4.51 | 4.01 | mmu-miR-151-5p | MIMAT0004536 | −3.75 | −5.20 |
| mmu-miR-148a-3p | MIMAT0000516 | 8.59 | 3.94 | mmu-miR-196b-5p | MIMAT0001081 | −3.51 | −5.05 |
| mmu-miR-193a-3p | MIMAT0000223 | 5.24 | 3.75 | mmu-miR-147-3p | MIMAT0004857 | −3.83 | −5.03 |
| mmu-miR-30b-5p | MIMAT0000130 | 2.73 | 3.74 | mmu-let-7i-5p | MIMAT0000122 | −2.02 | −4.99 |
| mmu-miR-200b-5p | MIMAT0004545 | 1.94 | 3.34 | mmu-miR-1195 | MIMAT0005856 | −2.30 | −4.97 |
| mmu-miR-677-5p | MIMAT0003451 | 2.31 | 3.17 | mmu-miR-96-5p | MIMAT0000541 | −3.41 | −4.62 |
| mmu-miR-154-3p | MIMAT0004537 | 1.72 | 2.96 | mmu-miR-669b-5p | MIMAT0003476 | −2.15 | −4.60 |
| mmu-miR-678 | MIMAT0003452 | 3.02 | 2.79 | mmu-miR-380-3p | MIMAT0000745 | −2.36 | −4.17 |
| mmu-miR-222-3p | MIMAT0000670 | 2.59 | 2.57 | mmu-miR-196a-5p | MIMAT0000518 | −5.16 | −4.02 |
| mmu-miR-203-3p | MIMAT0000236 | 6.86 | 2.52 | mmu-miR-375-3p | MIMAT0000739 | −2.74 | −3.99 |
| mmu-miR-331-3p | MIMAT0000571 | 3.13 | 2.44 | mmu-miR-181d-5p | MIMAT0004324 | −3.32 | −3.90 |
| mmu-miR-714 | MIMAT0003505 | 12.01 | 2.25 | mmu-miR-672-5p | MIMAT0003735 | −2.58 | −3.74 |
| mmu-miR-883b-3p | MIMAT0004851 | 1.84 | 1.94 | mmu-miR-654-3p | MIMAT0004898 | −2.43 | −3.74 |
| | | | | mmu-miR-1199-5p | MIMAT0005860 | −1.74 | −3.49 |
| | | | | mmu-miR-683 | MIMAT0003461 | −1.80 | −3.43 |
| | | | | mmu-miR-882 | MIMAT0004847 | −1.94 | −3.35 |
| | | | | mmu-miR-875-3p | MIMAT0004938 | −1.89 | −3.29 |
| | | | | mmu-miR-711 | MIMAT0003501 | −1.87 | −3.20 |
| | | | | mmu-miR-182-5p | MIMAT0000211 | −2.45 | −3.15 |
| | | | | mmu-miR-466f-5p | MIMAT0004881 | −2.23 | −3.13 |
| | | | | mmu-miR-764-3p | MIMAT0003895 | −3.50 | −2.85 |
| | | | | mmu-miR-504-5p | MIMAT0004889 | −2.16 | −2.62 |
| | | | | mmu-miR-17-5p | MIMAT0000649 | −4.07 | −2.61 |
| | | | | mmu-miR-1892 | MIMAT0007871 | −1.38 | −2.57 |
| | | | | mmu-miR-205-5p | MIMAT0000238 | −4.53 | −2.51 |
| | | | | mmu-miR-146b-5p | MIMAT0003475 | −1.79 | −2.46 |
| | | | | mmu-miR-301b-3p | MIMAT0004186 | −2.90 | −2.16 |
| | | | | mmu-miR-15b-5p | MIMAT0000124 | −4.14 | −1.92 |
| | | | | mmu-miR-450b-3p | MIMAT0003512 | −3.68 | −1.74 |

### Example 3—Tumor Suppressor miRNAs Selective Sort into Exosomes from Donor Tumor Cells

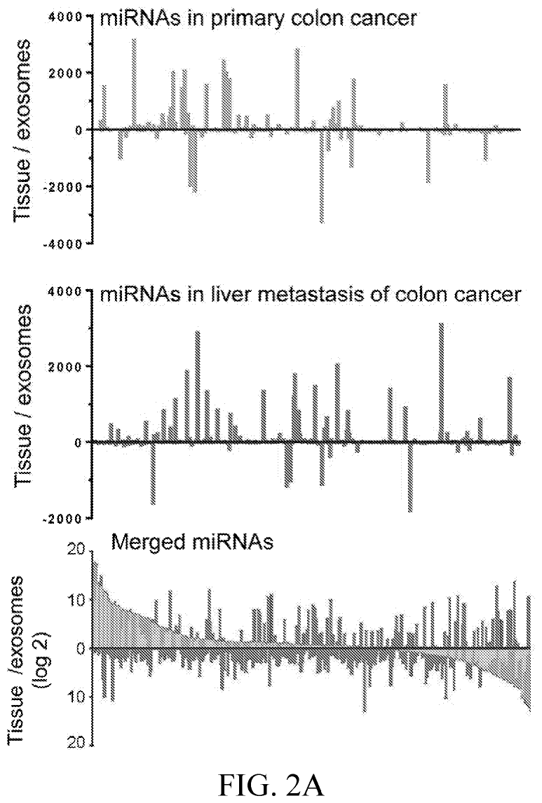

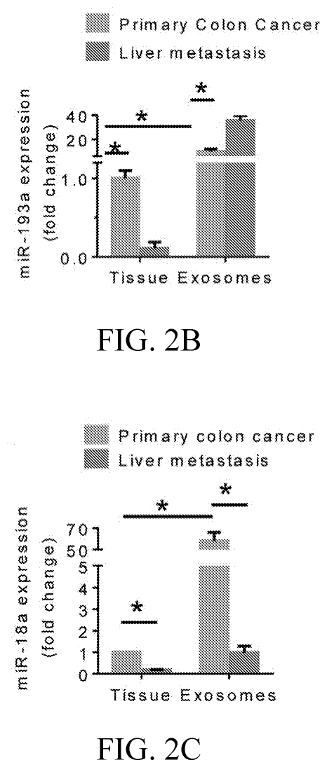

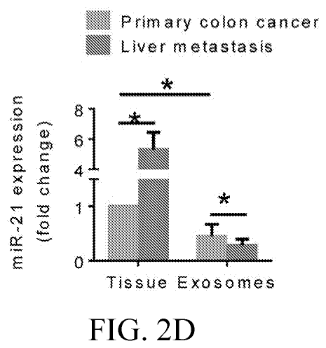

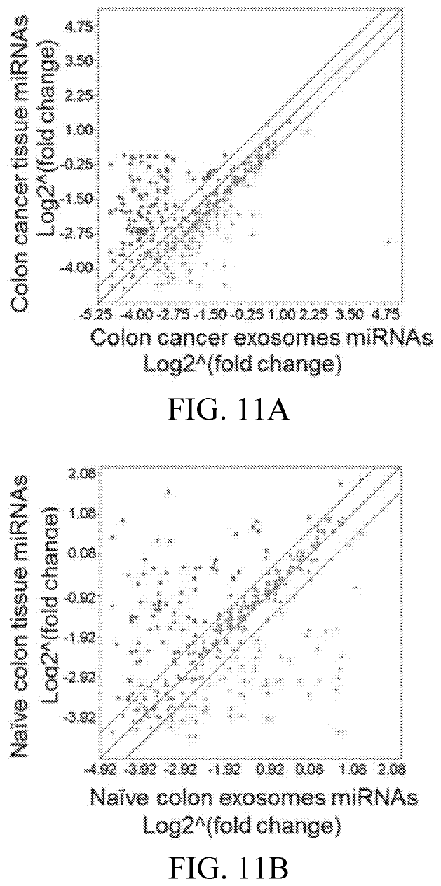

To determine whether the miRNA repertoires of exosomes differ from those of their donor cells, the profiles of miRNAs from exosomes and their donor cells were quantitatively analyzed. Scatter plot (FIGS. 11A-11B) results demonstrated a difference in exosomal RNA profiles from primary colon tumor (FIG. 11A) and naïve colon (FIG. 11B) from their donor tissues. The ratios of any given miRNA from exosomes and their donor cells were then calculated and are presented in FIG. 2A. The data suggested that miRNAs loaded into exosomes was not a passive process (FIG. 2A). 26.7% of miRNAs analyzed were higher and 47.5% of miRNAs were lower in primary colon tumor-derived exosomes than in exosomal donor tumor cells; 25.8% of miRNAs analyzed were presented in the exosomes and the exosomal donor cell at similar amounts (FIG. 2A, top panel). However, when CT26 colon tumor cells metastasize to the liver, the pattern of the CT26 tumor exosomal miRNA profile was altered (FIG. 2A, middle panel) in comparison to the pattern of the exosomal miRNAs expressed in the primary colon tumor. Some of the exosomal miRNAs retain the same patterns as indicated in the yellow color regardless of whether they are in the primary colon tumor or metastatic liver of colon cancer (FIG. 2A, bottom panel).

To investigate whether the level of tumor exosomal miRNA was sorted based on the miRNA biological function, the miRNA profiles were summarized (Table 3) based on miRNA oncogenic versus tumor suppressive effect. It was found that among three types of exosomes isolated from normal colon tissue, primary colon tumor tissue, and metastatic liver of colon tumor, metastatic CT26 colon-derived exosomes had the highest level of tumor suppressive miRNAs and the lowest level of oncogenic miRNAs. It was then investigated whether such a difference was determined by the level of miRNA expressed in the parent tissue. As summarized in Table 2, it was noticed that most of these miRNAs (miR-10a-5p, miR-193a-3p, miR-200b-5p, miR-222-3p) that were actively sorted into exosomes had tumor suppressive effects involving cell growth suppression; whereas, miRNAs (miR-196a/b, miR-181d-5p, miR-155-5p) that had oncogenic effects were retained in the tumor cells even through the levels of the oncogenic miRNAs were higher in their donor cells than in the exosomes. This assumption was further demonstrated with the data generated by reverse transcription-quantitative PCR (RT-qPCR) analysis of tumor suppressive miR-193a (FIG. 2B), miR18a (FIG. 2C), and oncogenic miR21 (FIG. 2D) as an example. The results indicate that the level of miR-18a and miR193a, in the exosomes from either primary colon tumor tissue or metastatic liver or colon tumor is higher than in their donor tumor tissue. In contrast, the level of oncogenic miR-21 was much higher in the primary colon tumor tissue and metastatic liver of colon tumor than in their exosomes. Collectively, those data indicate that oncogenic miRNAs are upregulated and tumor suppressive miRNAs are down regulated in the tumor, and this phenomenon is observed in metastatic liver of colon tumor. Sorting oncogenic miRNAs from exosomal donor cells into their exosomes is suppressed whereas sorting tumor suppressive miRNAs into exosomes is enhanced. Without wishing to be bound by any particular theory or mechanism, it is believed that such sorting can be one of mechanisms underlying tumor exosome mediated promotion of tumor progression.

TABLE 3
Differential distribution of miRNAs in exosomes and exosome donor cells (fold change).

| | Exosomes/tissue (log2) | | | | | Exosomes/tissue (log2) | | | |
|---|---|---|---|---|---|---|---|---|---|
| Higher in tumour | | Primary | | Biological | Lower in tumour | | Primary | | Biological |
| exo | Naive | cancer | Metastasis | effect | exo | Naive | cancer | Metastasis | effect |
| mmu-miR-10a-5p | −4.54 | −3.22 | −2.87 | TS/Onco | mmu-miR-423-5p | −0.07 | −0.42 | −7.49 | Onco |
| mmu-miR-126a-3p | 1.88 | −0.41 | −2.35 | TS/Onco | mmu-miR-301a-3p | 4.41 | 3.25 | 2.97 | Onco |
| mmu-miR-22-3p | −12.27 | −8.25 | −0.05 | TS | mmu-miR-33-5p | 1.87 | 1.31 | 1.24 | Onco/TS |
| mmu-miR-192-5p | −6.07 | −4.07 | −2.74 | TS | mmu-miR-9-5p | 1.10 | −1.15 | −3.78 | TS |
| mmu-miR-339-5p | −7.11 | −3.19 | 0.42 | TS | mmu-miR-151-5p | 1.02 | 4.98 | 7.91 | Onco |
| mmu-miR-148a-3p | −13.07 | −3.34 | −0.70 | TS | mmu-miR-196b-5p | 8.99 | −6.74 | −10.65 | Onco |
| mmu-miR-193a-3p | −7.01 | 7.58 | 7.77 | TS | mmu-miR-147-3p | −0.48 | −1.12 | −1.97 | TS |
| mmu-miR-30b-5p | −6.27 | −2.21 | −1.56 | TS | mmu-let-7i-5p | 0.25 | 3.36 | 5.10 | TS |
| mmu-miR-200b-5p | −9.94 | −5.57 | −4.53 | TS | mmu-miR-1195 | −0.30 | 0.15 | 0.46 | — |
| mmu-miR-677-5p | 0.49 | −1.05 | 7.76 | — | mmu-miR-96-5p | 1.43 | −1.10 | −0.90 | Onco |
| mmu-miR-154-3p | −3.93 | 2.01 | 5.43 | TS | mmu-miR-669b-5p | 0.31 | −2.57 | −5.07 | — |
| mmu-miR-678 | −5.82 | −1.09 | −1.76 | — | mmu-miR-380-3p | −0.07 | −1.88 | −3.46 | Onco |
| mmu-miR-222-3p | −2.42 | 3.64 | 8.62 | TS/Onco | mmu-miR-196a-5p | 7.97 | 5.36 | 3.78 | Onco |
| mmu-miR-203-3p | 0.03 | 2.24 | −1.65 | TS | mmu-miR-375-3p | 17.09 | 11.17 | 9.70 | Onco |
| mmu-miR-331-3p | −5.17 | −2.87 | −1.78 | TS | mmu-miR-181d-5p | 0.49 | −3.05 | −4.81 | Onco |
| mmu-miR-714 | 4.71 | 5.38 | 9.67 | — | mmu-miR-672-5p | 7.38 | 5.47 | 5.65 | — |
| mmu-miR-883b-3p | 0.95 | −4.49 | −7.50 | — | mmu-miR-654-3p | 5.88 | −2.97 | −5.51 | — |
| | | | | | mmu-miR-1199-5p | 2.32 | −0.44 | −1.69 | — |
| | | | | | mmu-miR-683 | 0.32 | −1.69 | −3.88 | — |
| | | | | | mmu-miR-882 | −0.61 | −2.23 | −1.87 | — |
| | | | | | mmu-miR-875-3p | 2.42 | 1.14 | 0.35 | — |
| | | | | | mmu-miR-711 | 1.21 | −4.46 | −6.21 | Onco |
| | | | | | mmu-miR-182-5p | 0.13 | −1.07 | −1.81 | Onco |
| | | | | | mmu-miR-466f-5p | −0.49 | 1.12 | −2.74 | — |
| | | | | | mmu-miR-764-3p | 2.50 | −1.08 | −3.64 | — |
| | | | | | mmu-miR-504-5p | 6.17 | −1.32 | 1.77 | TS |
| | | | | | mmu-miR-17-5p | 0.17 | −3.71 | −7.81 | Onco |
| | | | | | mmu-miR-1892 | 1.39 | −4.07 | −8.27 | — |
| | | | | | mmu-miR-205-5p | 3.82 | −2.84 | −1.04 | Onco/TS |
| | | | | | mmu-miR-146b-5p | 0.01 | −1.12 | −2.30 | Onco |
| | | | | | mmu-miR-301b-3p | 0.99 | −2.87 | −3.56 | Onco |
| | | | | | mmu-miR-15b-5p | 0.64 | −2.30 | −3.02 | Onco |
| | | | | | mmu-miR-450b-3p | −4.20 | −3.21 | −3.50 | — |

Onco, oncogenic miRNAs;
TS, tumour suppressive miRNAs;
—, unknown.

### Example 4—Microenvironment has an Effect on the Composition of Tumor Exosomal miRNA Profiles

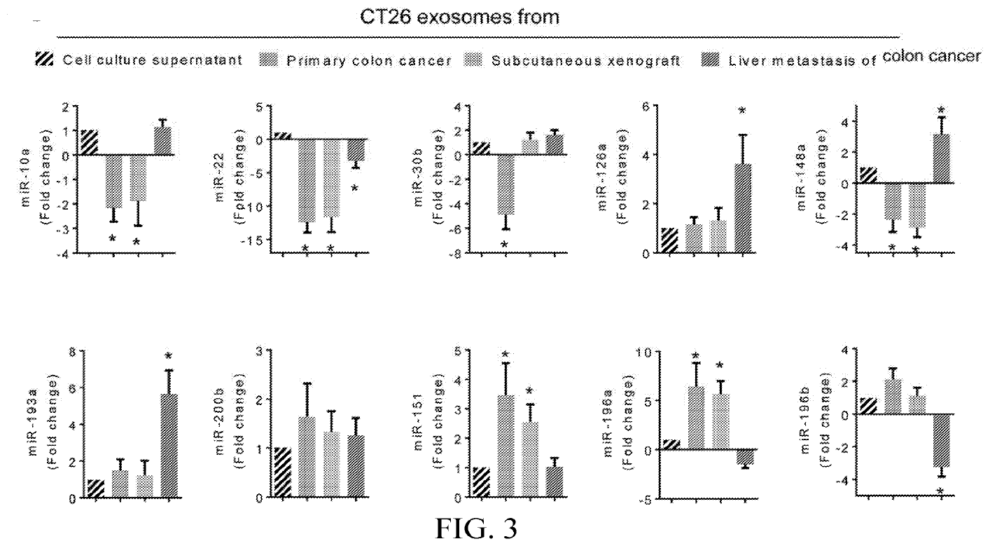

Published data shows the biological activities of tumor EVs using in vitro cultured tumor cell derived EVs. This may not accurately represent the case for tumor EV release from tumor tissue because multiple factors derived from tumor tissue have a remarkable effect on the composition of tumor EVs, and those factors do not exist in culture medium. In this regard, the levels of selected miRNAs (FIG. 3) present in exosomes released from in vitro cultured CT26 cells (culture medium environment), from primary colon cancer, CT26 subcutaneous xenograft, and from metastatic CT26 tumor isolated from mouse liver were compared. The results generated from qPCR showed that miR126a, miR148a, and miR-193a are significantly higher in the exosomes released from metastatic CT26, but not from primary colon cancer or subcutaneous xenograft. However, miR22, miR-196a and miR-196b were decreased in the exosomes from metastatic liver of colon tumor (FIG. 3) compared with exosomes from in vitro cultured CT26. These changes were specific as other miRNAs including miR-10a, miR-30b, miR200b, and miR-151 have no change regardless if the origin is from the exosomes of cultured tumor cells or metastatic CT26, indicating that the microenvironment has an effect on the composition of exosomes miRNA profile.

### Example 5—MiR-193 Suppresses Mouse Colon Cancer Progression by Directly Targeting Caprin1

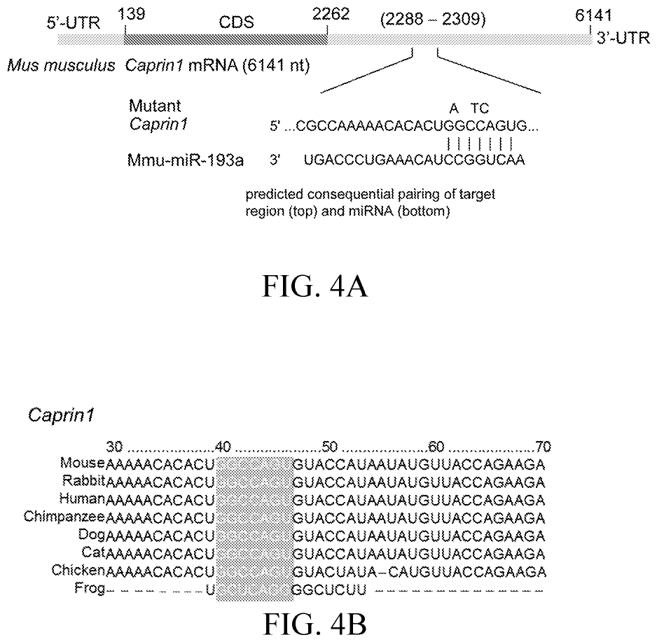

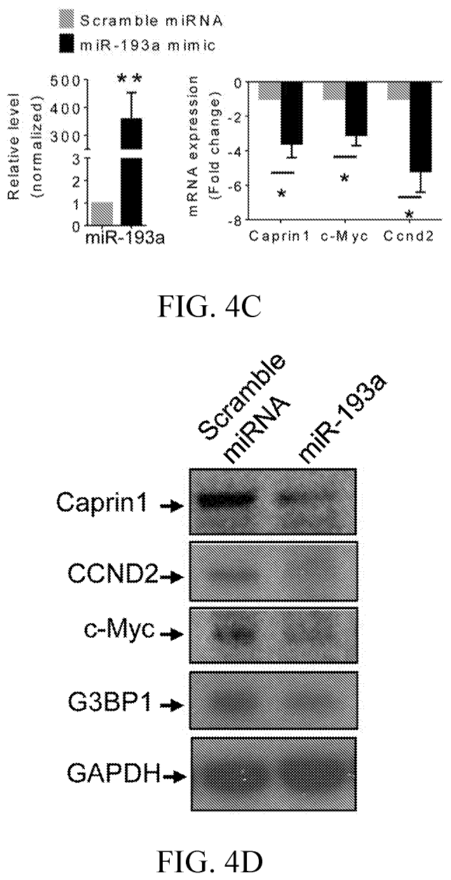

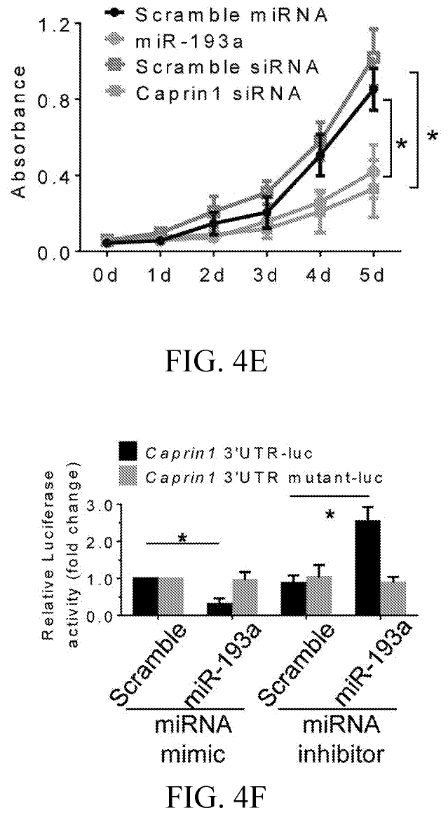

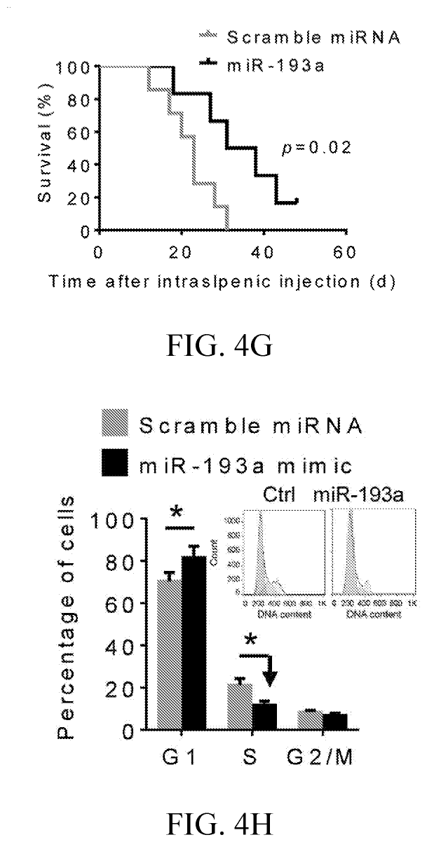

It was further hypothesized that exporting tumor suppressive miRNA such as miR-193a, as an example in this study, from exosome donor cells into exosomes is a benefit for colon cancer metastasis to the liver. miRNA databases were first searched for potential miR-193a targets that may contribute or promote tumor progression. Three public miRNA databases (TargetScan, Pictar, and MicroRNA) all predicted that cell cycle-associated protein Caprin1 might be a target for miR-193a (FIG. 4A), and the 3′-UTR of Caprin1 contains a highly conserved binding site from position 2288 to 2309 for miR-193a (FIGS. 4A-4B). To determine whether miR-193a could target Caprin1 in colon cancer cells, the mouse mature miR-193a mimic was transfected into CT26 cells. The CT26 cells overexpressing miR-193a (FIG. 4C, left) had a significantly down-regulated Caprin1 mRNA expression (FIG. 4C, right panel) as well as Caprin1 protein expression (FIG. 4D). It was also found that Ccnd2 and c-myc, which are regulated by Caprin1 and are also decreased as a result of miR-193a treatment (FIG. 4C-4D). The impact of miR-193a overexpression on the inhibition of cell proliferation was further confirmed by the Caprin1 siRNA knockdown in CT26 colon cancer cells (FIG. 4E). To ascertain the direct effect of miR-193a on Caprin1, a mutant construct that would disrupt the predicted miR-193a binding site was generated from pEZX-MT01-Caprin1 containing a full length 3′UTR of Caprin1 mRNA (Gene Accession: NM_001111289). A luciferase reporter assay was performed by co-transfecting a vector containing Caprin1 3′UTR fused luciferase and miR-193a or control miRNA as a negative control. Overexpression of miR-193a decreased the luciferase activity of the reporter with the 3′UTR of Caprin1 by about 56% in CT26 cells (FIG. 4F). However, mutation that disrupted the binding site for miR-193a entirely restored luciferase activity. Moreover, overexpression of anti-sense miR-193a (miRNA inhibitor) caused induction of luciferase and no inductive effect of anti-sense miR-193a on the activity of the reporter with a mutant 3′UTR of Caprin1 was detected (FIG. 4F). These results demonstrate that Caprin1 is a target of miR-193a in colon cancer cells. The tumor suppression role of miR-193a was further supported by the fact that overexpression of miR-193a inhibited CT26 cell proliferation and significantly prolonged survival of colon cancer bearing mice (FIG. 4G). Cell cycle assessment suggested that miR-193 causes S phase arrest in cell cycle (FIG. 4H).

### Example 6—Major Vault Protein (MVP) Regulates the Loading of miR-193a from Tumor Cell to Exosomes

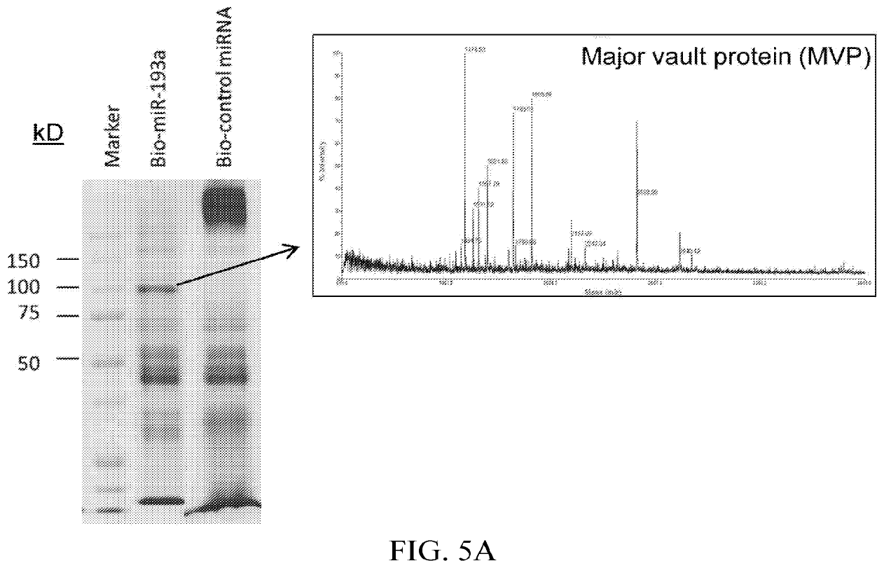

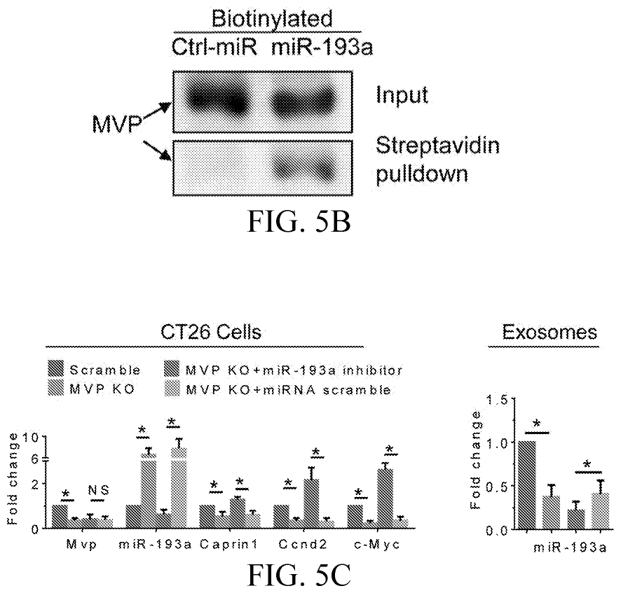

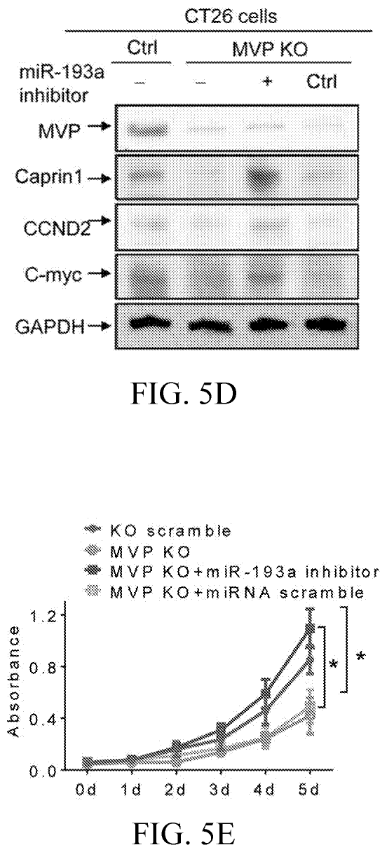

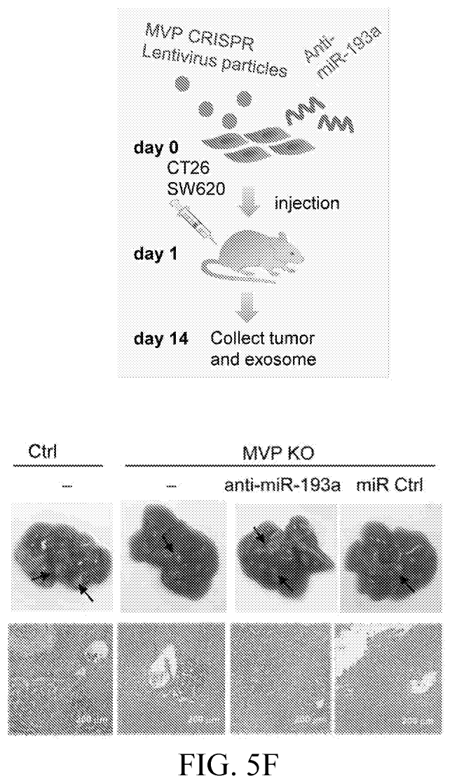

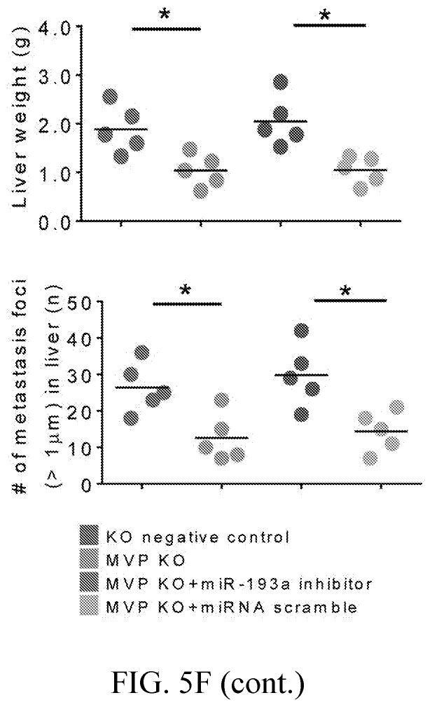

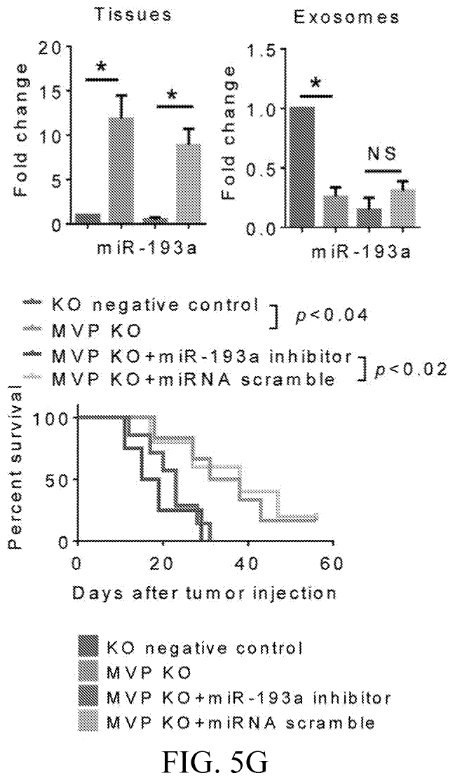

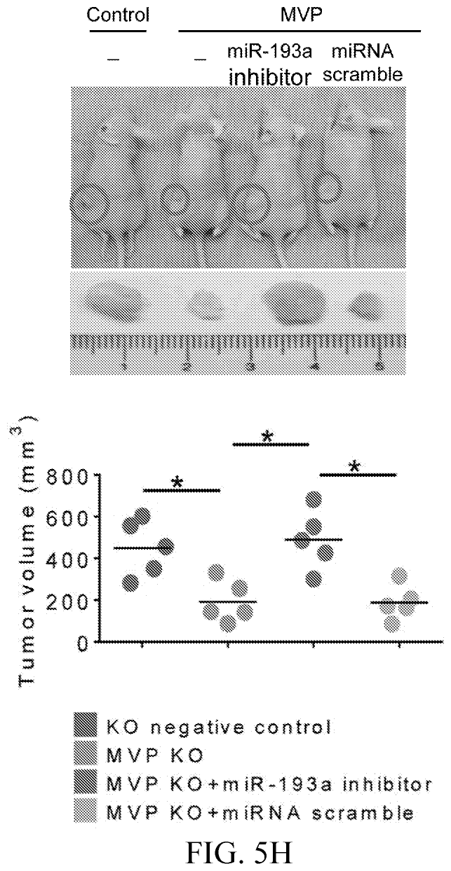

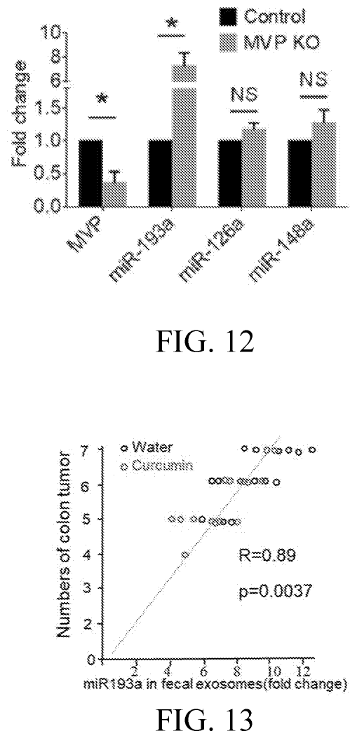

Although the miRNA repertoires of exosomes differ from those of their donor cells, the explanation or mechanism how this occurs is still unknown. It was hypothesized that host factor(s) might play a role in miRNA sorting from exosomal donor cells to their exosomes. To test the hypothesis, biotin labeled miR-193a complex was isolated from exosomal lysates using streptavidin beads. A typical staining pattern of the Bio-miR-193a complex obtained from CT26 exosomal extracts on sodium dodecyl sulfate polyacrylamide gel electrophoresis (SDS-PAGE) is shown in FIG. 5A, left panel. In-gel digestion-MALDI-TOF mass spectrometry (MS) analysis was carried out for identification of proteins that are specific presenting in Bio-miR-193a complex sample but not in the control bio-miRNA complex. Major vault protein (MVP) was subsequently identified as a potential miR-193a binding protein by MS (FIG. 5A, right panel) and this interaction was verified by Western blot (FIG. 5B). CT26 cells transfected with MVP knock out (KO) MVP sgRNA CRISPR Lentivirus (>1×10⁷IU/ml) have low levels of MVP detected (FIGS. 5C-5D), but the level of miR-193a is increased in the cells (FIG. 5C, left panel). This finding is inversely correlated with the levels of miR-193a in the exosomes (FIG. 5C, right panel). The accumulation of miRNA in the MVP KO CT26 cells is miR-193a specific since no change of miR-126a was observed due to MVP KO (FIG. 12). Collectively, these data suggest that miR-193a sorted into exosomes is MVP dependent. To further understand the effects of MVP KO on the cells, cell proliferation of MVP KO CT26 cells was compared with scramble KO control cells. MVP KO led to tumor cell proliferation repression (FIG. 5E). This result agreed with cyclin D2 (CCND2) and c-myc being decreased at the both transcriptional (FIG. 5C, left panel) and protein levels (FIG. 5D) Inhibition of miR-193a expression reversed the effects of MVP KO on repressive expression of Caprin1, CCND2 and c-myc (FIG. 5D), eventually leading to induction of cell growth (FIG. 5E). The results generated from a mouse colon cancer model with liver metastasis further supports that miR-193a exported via exosomes by MVP leads to promotion of tumor progression (FIG. 5F). MVP KO in CT26 colon tumor cells resulted in the inhibition of liver metastasis of colon cancer (FIG. 5F, middle and right panels), and this result correlates with a miR-193a increase in CT26 cells and decrease in exosomes (FIG. 5G, left panel). Knock down of miR-193a expression by miR-193a inhibitor enhanced tumor metastasis in liver and decreased survival of colon cancer bearing mice (FIG. 5G, right panel).

The MVP mediated promotion of tumor progression through miR-193a is also demonstrated by subcutaneous injection of human colon cancer SW620 cells into nude mice (FIG. 5H). Mice injected subcutaneously with SW620 cells showed remarkable tumor growth over 14 days. However, mice injected subcutaneously with MVP KO SW620 cells have a much slower tumor growth rate. This repression on tumor growth was partially reversed by knocking down the expression of miR-193a (FIGS. 5H-5I). Analysis of miR-193 levels in tumor by qPCR indicated that MVP KO caused the accumulation of miR-193a in cells (FIG. 5I, left panel) with a concomitant decrease of miR-193a in the exosomes (FIG. 5I, right panel). Collectively, these data indicate that MVP regulates miR-193a sorting into exosomes and accumulation of miR-193a in the exosomal donor cells as a result of KO MVP is detrimental to tumor cells; whereas reduction of miR-193a by MVP dependent sorting into exosomes leads to tumor cell proliferation and a faster cell cycle, eventually enhancing tumor cell growth and metastasis.

### Example 7—Higher Levels of Exosomal miR-193a in the Peripheral Blood of Colon Cancer Patients Lead to More Aggressive Disease

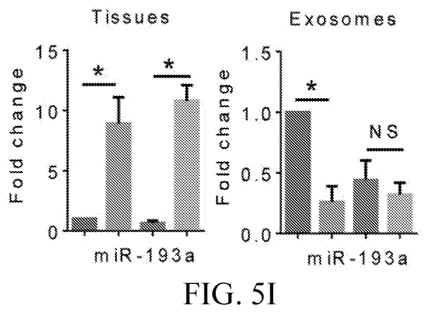

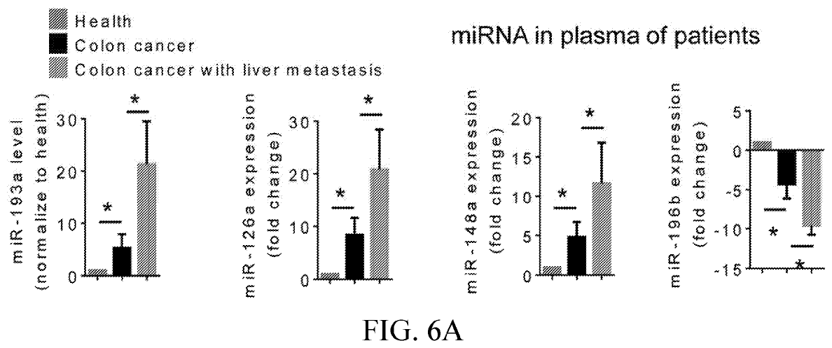

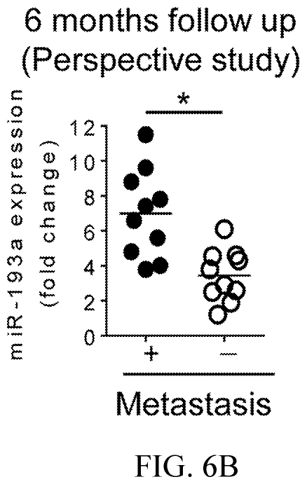

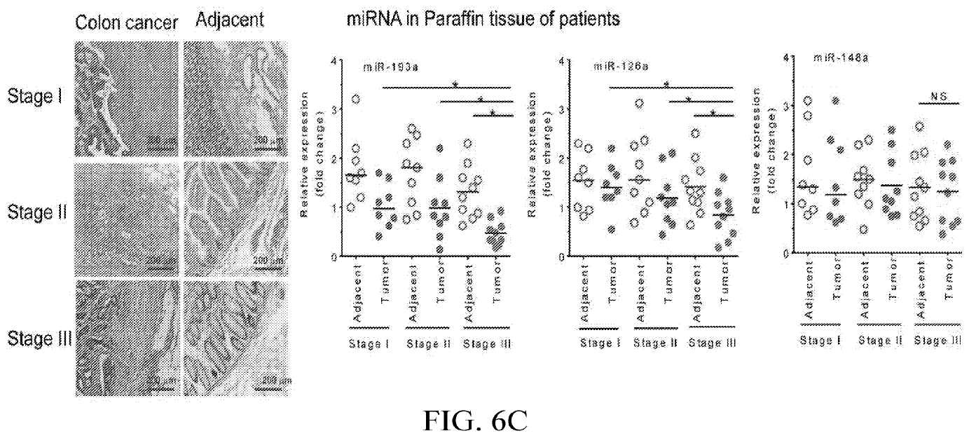

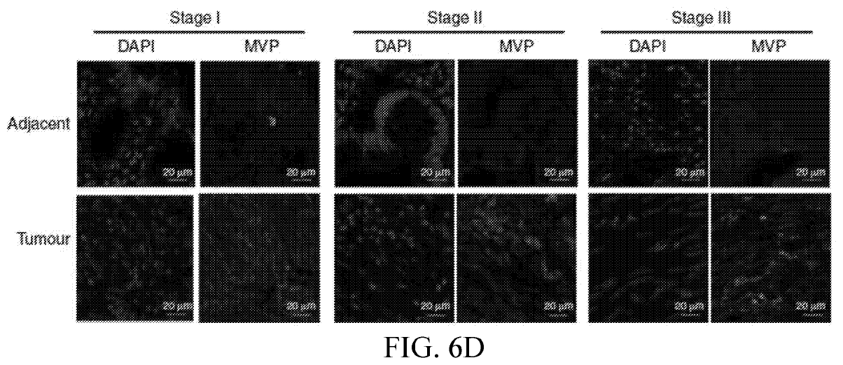

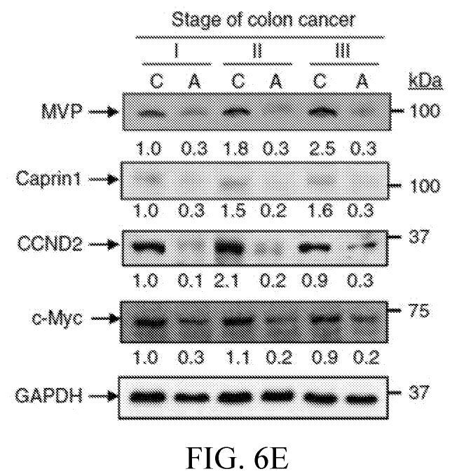

To further determine whether our finding as described above can be translated into clinical application, three up-regulated (miR-193a, miR-126 and miR-148a) and one down-regulated miRNA (miR-196b) found in the exosomes isolated from metastatic liver in a mouse colon cancer were analyzed in colon cancer patients by qPCR. Twenty-five colon cancer patients without metastasis and fifteen colon cancer patients with liver metastasis were enrolled. Overall, the exosomes isolated from peripheral plasma of colon cancer patients exhibited higher levels of miR-193a, miR-126 and miR-148a and lower levels of miR-196b compared to healthy controls (FIG. 6A). Furthermore, the exosomes isolated from plasma of colon cancer patients with liver metastasis displayed higher levels of miR-193a, miR-126 and miR-148a and lower levels of miR-196b compared to colon cancer patients having no metastasis (FIG. 6A). The liver metastasis incidence was further investigated with a six month follow-up after primary diagnosis. This perspective study indicated that colon cancer patients with high levels of miR-193a in exosomes from peripheral blood have a higher risk of metastasis (FIG. 6B). Colon cancer stages of patients were classified by H&E staining with an estimate of the levels of infiltration of a particular cancer (FIG. 6C, left panel). The miRNAs expressed in tumor tissue and adjacent normal tissue (used as a control) were furthermore analyzed by qPCR. The level of miR-193a in the tumor was lower than in adjacent tissue in all three stages of colon cancer. The down-regulation of miR-193a in tumor tissue was stage-dependent (FIG. 6C, second panel from left). MiR-126a down-regulation only appeared in stage III of colon cancer comparing to adjacent normal tissue (FIG. 6C, third panel from left). Although miR-148a increased in peripheral plasma of colon cancer patients, no difference in miR-148a level between tumor tissue and adjacent tissue was evident (FIG. 6C, right panel). Induction of MVP in colon cancer tissue is stage dependent and supported by confocal immunostaining (FIG. 6D) and immunoblotting results (FIG. 6E). Along with the increase of MVP, the induction of Caprin1, CCDN1 and C-myc was detected at the protein level (FIG. 6E).

### Discussion of Examples 1-7

Cancer EVs including exosomes play a role in promoting tumor progression. Most published data show the biological activities of tumor-derived EVs using cultured tumor cell-derived EVs which may not accurately reflect the case for tumor cell-derived EVs released from tumor tissue. A major challenge for studying the biological activities of tumor cell-derived EVs released from tumor tissue is to develop methodology to isolate EVs from tumor cells when they are mixed with other EVs. In this study, using a biotin-streptavidin based detection approach we demonstrated that our methodology is suitable for isolation of tumor cell-derived EVs from tumor tissue. The fact that qPCR results generated with the tumor tissue-derived exosomal miRNA agreed with the data from exosomal miRNA isolated from peripheral blood of CT26 tumor bearing mice but not naïve mice further warrants using this technology for future research in the EV field. Moreover, this conclusion is not only supported from data using mouse colon cancer models but also by the data generated from exosomes isolated from peripheral blood of colon cancer patients. Therefore, this approach will open up a new avenue for isolation, characterization, and functional analysis of any subpopulation of EVs. In addition, the results generated from this approach will provide a standard reference for evaluating the level of potential contaminants in the EVs isolated from tissue with a non-biotin-streptavidin based approach such as collagenase digested tissue, followed by a standard differential centrifugation method for isolation of tissue exosomes.

More importantly, in the above-described study, the approach led to the new finding that (1) tumor suppressor miRNAs are sorted into tumor exosomes whereas oncogenic miRNAs remain in the tumor cells; and (2) in severe disease higher levels of tumor suppressor miRNAs are present in the tumor exosomes. This conclusion is also supported by the data generated from exosomes isolated from peripheral blood of colon cancer patients. Therefore, this finding provides a solid foundation for further investigating the molecular mechanism(s) underlying how tumor cells selectively sort tumor suppressor miRNA out of cell. The findings with the tumor suppressor miR-193a demonstrates this phenomenon.

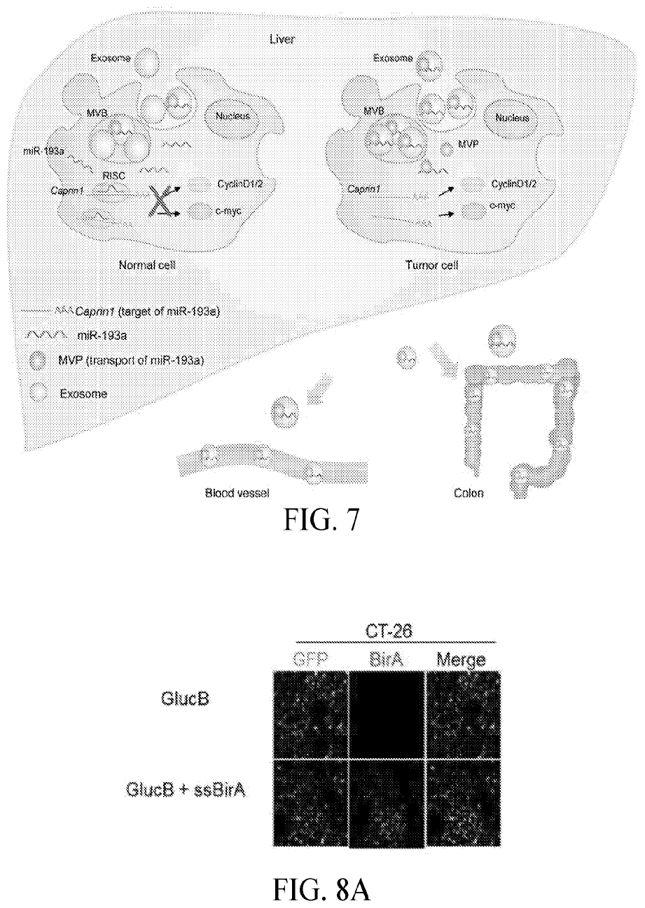

MVP is overexpressed in multidrug-resistant cancer cells. This protein mediates transport of nucleic acids, proteins and drugs between the nucleus and cytoplasm. In the foregoing study, it was demonstrated that MVP transports miR19a from tumor cells to exosomes. As summarized in FIG. 7, MVP binds to tumor suppressor miR-193a forming a MVP protein-miR-193a complex. Subsequently this complex is packed into exosomes leading to the reduction of cytoplasmic miR-193a. The fact that MVP knockout causes miR-193a accumulation in cells instead of exosomes further supports this finding. The study demonstrates that accumulation of miR-193a in the tumor cell leads to inhibition of tumor growth. The data presented in this study indicates that miR-193a targets the 3′UTR of Caprin-1 mRNA leading to inhibition of production of caprin-1 protein. Caprin-1 is known to regulate the cell cycle and cell proliferation. Caprin-1 directly binds to mRNAs for G3BP1 protein, c-Myc and cyclin D2 through its carboxy-terminal RGG-rich region. We found that higher levels of miR-193a in tumor cells causes cell cycle S arrest and cell proliferation repression through reduction of caprin-1 expression. The impact of miR-193a mediated interrupt of the caprin-1/G3BP-1/c-Myc/Cyclin D2 complex could be a potential target for anti-cancer therapeutic applications.

Besides miR-193a targeting of caprin-1, other potential molecules could be targeted by miR193a, such as GTPase Rab27b which regulates exosome biogenesis. Accumulation of miR-193a in tumor cells may also inhibit the release of exosomes via miR-193a-3p mediated targeting of GTPase Rab27b which has been reported by other groups. Therefore, it is speculated that accumulation of miR-193a in tumor cells by knockdown of MVP may prevent exosome release, thus contributing to inhibition of tumor progression as well. It is well-known that the tumor microenvironment has dramatic effects on the outcome of tumor growth. Whether the tumor microenvironment affects the composition of the tumor exosome miRNA profile has not been fully investigated. In the foregoing study, it was shown that the levels of tumor exosomal miRNAs from metastatic liver of colon cancer is different from the profile generated from an orthotopic cecum tumor model, subcutaneous xenograft tumor model or in vitro cell culture derived miRNA profile. This result implies that these significantly high levels of miRNAs present in the exosomes released from metastatic liver of colon cancer are potential candidates for prognosis and diagnosis of colon cancer, in particular for liver metastasis. Liver metastasis of colon cancer processes depend on multiple interactions between cancer cells in the tumor and host derived cells in the microenvironment in both the primary tumor and secondary organ; however, they often occur undetected in the patient. Thus, in spite of its devastating impact, metastasis continues to be diagnosed in its final stage when little can be done. Identifying the exosomal miRNAs representing liver metastasis could lead to the development of more accurate methods of early detection and intervention in the metastatic process.

### Example 8—Evaluation of miR-193a in Fecal Samples

As shown in FIG. 13, higher levels of exosomal miR193a in mouse feces also correlated with increased numbers of colon tumors, and it was observed that drinking curcumin offered some protection against that colon effect. Briefly, in these experiments, 7-week old C57/BL6 male mice (n=15) were injected intraperitoneally with Azoxymethane (AOM) solution (10 mg/kg body weight of mice). On day 7, the 2% dextran sulfate sodium (DSS) solution (wt/vol) was given in drinking water with/without curcumin (20 mg/kg body weight of mice) for 5 days. On day 14, 2% DSS drinking water was replaced with autoclaved drinking water with/without curcumin (20 mg/kg body weight of mice) and leave for 14 days. On days 28-33 and days 48-54, repeat DSS treatment as above. On day 100, mice were sacrificed, and the number of colon tumor was accounted and feces collected for exosomes purification and qPCR of exosomes miR193a was carried out. In FIG. 13, each data represents the mean±SEM for 15 mice from each group with Regression analysis.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Teng, Y. et al. Grapefruit-derived nanovectors deliver miR-18a for treatment of liver metastasis of colon cancer by induction of M1 macrophages. Oncotarget, doi:10.18632/oncotarget.8361 (2016).
2. Xiang, X. et al. miR-155 promotes macroscopic tumor formation yet inhibits tumor dissemination from mammary fat pads to the lung by preventing EMT. Oncogene 30, 3440-3453, doi:10.1038/onc.2011.54 (2011).
3. Liu, Y. et al. COP9-associated CSN5 regulates exosomal protein deubiquitination and sorting. The American journal of pathology 174, 1415-1425, doi:10.2353/ajpath.2009.080861 (2009).
4. Tkach, M. & Thery, C. Communication by Extracellular Vesicles: Where We Are and Where We Need to Go. Cell 164, 1226-1232, doi:10.1016/j.cell.2016.01.043 (2016).
5. Thery, C. Cancer: Diagnosis by extracellular vesicles. Nature 523, 161-162, doi:10.1038/nature14626 (2015).
6. Bobrie, A. & Thery, C. Exosomes and communication between tumours and the immune system: are all exosomes equal? Biochemical Society transactions 41, 263-267, doi:10.1042/BST20120245 (2013).
7. Bobrie, A., Colombo, M., Krumeich, S., Raposo, G. & Thery, C. Diverse subpopulations of vesicles secreted by different intracellular mechanisms are present in exosome preparations obtained by differential ultracentrifugation. Journal of extracellular vesicles 1, doi:10.3402/jev.v1i0.18397 (2012).
8. Thery, C., Ostrowski, M. & Segura, E. Membrane vesicles as conveyors of immune responses. Nature reviews. Immunology 9, 581-593, doi:10.1038/nri2567 (2009).
9. Peinado, H. et al. Melanoma exosomes educate bone marrow progenitor cells toward a pro-metastatic phenotype through MET. Nature medicine 18, 883-891, doi:10.1038/nm.2753 (2012).
10. Bobrie, A. et al. Rab27a supports exosome-dependent and -independent mechanisms that modify the tumor microenvironment and can promote tumor progression. Cancer research 72, 4920-4930, doi:10.1158/0008-5472.CAN-12-0925 (2012).
11. Trajkovic, K. et al. Ceramide triggers budding of exosome vesicles into multivesicular endosomes. Science 319, 1244-1247, doi:10.1126/science.1153124 (2008).
12. Lai, C. P., Tannous, B. A. & Breakefield, X. O. Noninvasive in vivo monitoring of extracellular vesicles. Methods in molecular biology 1098, 249-258, doi:10.1007/978-1-62703-718-1_19 (2014).
13. Zomer, A. et al. Exosomes: Fit to deliver small RNA. Communicative & integrative biology 3, 447-450, doi:10.4161/cib.3.5.12339 (2010).
14. Ridder, K. et al. Extracellular vesicle-mediated transfer of functional RNA in the tumor microenvironment. Oncoimmunology 4, e1008371, doi:10.1080/2162402X.2015.1008371 (2015).
15. Lai, C. P. et al. Dynamic biodistribution of extracellular vesicles in vivo using a multimodal imaging reporter. ACS nano 8, 483-494, doi:10.1021/nn404945r (2014).
16. Teng, Y. et al. Grapefruit-derived nanovectors deliver miR-18a for treatment of liver metastasis of colon cancer by induction of M1 macrophages. Oncotarget, doi:10.18632/oncotarget.8361 (2016).
17. Hoshino, A. et al. Tumour exosome integrins determine organotropic metastasis. Nature 527, 329-335, doi:10.1038/nature15756 (2015).
18. Costa-Silva, B. et al. Pancreatic cancer exosomes initiate pre-metastatic niche formation in the liver. Nature cell biology 17, 816-826, doi:10.1038/ncb3169 (2015).
19. 16 Deng, Z. B. et al. Exosome-like nanoparticles from intestinal mucosal cells carry prostaglandin E2 and suppress activation of liver NKT cells. Journal of immunology 190, 3579-3589, doi:10.4049/jimmunol.1203170 (2013).
20. Deng, Z. B. et al. Adipose tissue exosome-like vesicles mediate activation of macrophage-induced insulin resistance. Diabetes 58, 2498-2505, doi:10.2337/db09-0216 (2009).
21. Zhao, Y. N., He, D. N., Wang, Y. D., Li, J. J. & Ha, M. W. Association of single nucleotide polymorphisms in the MVP gene with platinum resistance and survival in patients with epithelial ovarian cancer. Oncology letters 11, 2925-2933, doi:10.3892/ol.2016.4311 (2016).
22. Balan, S. et al. Major vault protein (MVP) gene polymorphisms and drug resistance in mesial temporal lobe epilepsy with hippocampal sclerosis. Gene 526, 449-453, doi:10.1016/j.gene.2013.05.067 (2013).
23. Lu, C. & Shervington, A. Chemoresistance in gliomas. Molecular and cellular biochemistry 312, 71-80, doi:10.1007/s11010-008-9722-8 (2008).
24. Yan, X. D., Li, M., Yuan, Y., Mao, N. & Pan, L. Y. Biological comparison of ovarian cancer resistant cell lines to cisplatin and Taxol by two different administrations. Oncology reports 17, 1163-1169 (2007).
25. Sisodiya, S. M. et al. Vascular colocalization of P-glycoprotein, multidrug-resistance associated protein 1, breast cancer resistance protein and major vault protein in human epileptogenic pathologies. Neuropathology and applied neurobiology 32, 51-63, doi:10.1111/j.1365-2990.2005.00699.x (2006).
26. Wu, Y., Zhu, J., Huang, X. & Du, Z. Crystal structure of a dimerization domain of human Caprin-1: insights into the assembly of an evolutionarily conserved ribonucleoprotein complex consisting of Caprin-1, FMRP and G3BP1. Acta Crystallogr D Struct Biol 72, 718-727, doi:10.1107/S2059798316004903 (2016).
27. Qiu, Y. Q. et al. Targeting a ribonucleoprotein complex containing the caprin-1 protein and the c-Myc mRNA suppresses tumor growth in mice: an identification of a novel oncotarget. Oncotarget 6, 2148-2163, doi:10.18632/oncotarget.3236 (2015).
28. Solomon, S. et al. Distinct structural features of caprin-1 mediate its interaction with G3BP-1 and its induction of phosphorylation of eukaryotic translation initiation factor 2alpha, entry to cytoplasmic stress granules, and selective interaction with a subset of mRNAs. Molecular and cellular biology 27, 2324-2342, doi:10.1128/MCB.02300-06 (2007).
29. Deng, Z. et al. Tumor cell cross talk with tumor-associated leukocytes leads to induction of tumor exosomal fibronectin and promotes tumor progression. The American journal of pathology 180, 390-398, doi:10.1016/j.ajpath.2011.09.023 (2012).
30. Teng Y, Ren Y, Hu X, Mu J, Samykutty A, Zhuang X, Deng Z, Kumar A, Zhang L, Merchant M L, Yan J, Miller D M, Zhang H G. MVP-mediated exosomal sorting of miR-193a promotes colon cancer progression. Nature communications. 2017; 8:14448. doi: 10.1038/ncomms14448. PubMed PMID: 28211508; PMCID: PMC5321731.
31. Deng Z, Mu J, Tseng M, Wattenberg B, Zhuang X, Egilmez N K, Wang Q, Zhang L, Norris J, Guo H, Yan J, Haribabu B, Miller D, Zhang H G. Enterobacteria-secreted particles induce production of exosome-like S1P-containing particles by intestinal epithelium to drive Th17-mediated tumorigenesis. Nature communications. 2015; 6:6956. doi: 10.1038/ncomms7956. PubMed PMID: 25907800; PMCID: PMC4410277.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A method for diagnosing a cancer in a subject, comprising:
providing a biological sample from the subject;
isolating exosomes from the biological sample;
identifying an amount of one or more tumor suppressor miRNAs in the exosomes; and
comparing the amount of the one or more tumor suppressor miRNAs in the exosomes, if present, to a control level of the one or more tumor suppressor miRNAs, wherein the subject is diagnosed as having cancer or a risk thereof if there is a measurable difference in the amount of the one or more tumor suppressor miRNAs in the exosomes as compared to the control level.

2. The method of claim 1, wherein the one or more tumor suppressor miRNAs comprise one or more miRNAs selected from the miRNAs of Table 2.

3. The method of claim 1, wherein the one or more tumor suppressor miRNAs comprises miR-193a.

4. The method of claim 1, wherein the cancer is selected from the group consisting of colon cancer and liver cancer.

5. The method of claim 1, wherein the cancer is a primary cancer or a secondary cancer.

6. The method of claim 1, wherein the biological sample comprises blood, plasma, serum, or feces.

7. The method of claim 1, wherein the subject is human.

8. The method of claim 5, wherein the subject has cancer.

9. The method of claim 1, wherein identifying the one or more tumor suppressor miRNAs in the exosomes comprises identifying the one or more tumor suppressor miRNAs using polymerase chain reaction (PCR) or microarray analysis.

10. The method of claim 1, wherein identifying the one or more tumor suppressor miRNAs comprises providing a probe for selectively binding each of the one or more miRNAs.

11. The method of claim 1, further comprising selecting a treatment or modifying a treatment for the cancer based on the identification of the one or more tumor suppressor miRNAs in the exosomes.

12. A method for determining whether to initiate or continue prophylaxis or treatment of a cancer in a subject, comprising:
providing a series of biological samples over a time period from the subject;
isolating exosomes from each biological sample in the series of biological samples;
identifying an amount of one or more tumor suppressor miRNAs in the exosomes isolated from each biological sample of the series of biological samples; and
comparing any measurable change in the amounts of the one or more tumor suppressor miRNAs in the exosomes in each biological sample of the series of biological samples to thereby determine whether to initiate or continue the prophylaxis or therapy of the cancer.

13. The method of claim 12, wherein the one or more tumor suppressor miRNAs comprise one or more miRNAs selected from the miRNAs of Table 2.

14. The method of claim 13, wherein the one or more tumor suppressor miRNAs comprises miR-193a.

15. The method of claim 12, wherein the cancer is selected from the group consisting of colon cancer and liver cancer.

16. The method of claim 12, wherein the cancer is a primary cancer or a secondary cancer.

17. The method of claim 12, wherein the biological sample comprises blood, plasma, serum, or feces.

18. The method of claim 12, wherein the series of biological samples comprises a first biological sample collected prior to initiation of the prophylaxis or treatment for cancer and a second biological sample collected after initiation of the prophylaxis or treatment.

19. The method of claim 12, wherein the series of biological samples comprises a first biological sample collected prior to onset of the cancer and a second biological sample collected after the onset of the cancer.

20. The method of claim 12, wherein identifying the one or more tumor suppressor miRNAs comprises providing a probe for selectively binding each of the one or more miRNAs.


SEQUENCE LISTING (17 sequences)

SEQ ID NO: 1 - DNA, 19 bp, Artificial Sequence
  Artificially synthesized oligonucleotide primer
agacgagtggctgtttgag

SEQ ID NO: 2 - DNA, 22 bp, Artificial Sequence
  Artificially synthesized oligonucleotide primer
cagagcttggttctgtttgatg

SEQ ID NO: 3 - DNA, 21 bp, Artificial Sequence
  Artificially synthesized oligonucleotide primer
cttatggcacaaatgcaaggg

SEQ ID NO: 4 - DNA, 23 bp, Artificial Sequence
  Artificially synthesized oligonucleotide primer
catgttctgggtagggttcatag

SEQ ID NO: 5 - DNA, 21 bp, Artificial Sequence
  Artificially synthesized oligonucleotide primer
gaaggacatccaaccgtacat

SEQ ID NO: 6 - DNA, 20 bp, Artificial Sequence
  Artificially synthesized oligonucleotide primer
ttcatggccagaggaaagac

SEQ ID NO: 7 - DNA, 19 bp, Artificial Sequence
  Artificially synthesized oligonucleotide primer
gcgatcagctctcctgaaa

SEQ ID NO: 8 - DNA, 21 bp, Artificial Sequence
  Artificially synthesized oligonucleotide primer
gcagaaagaacacagggaaag

SEQ ID NO: 9 - DNA, 20 bp, Artificial Sequence
  Artificially synthesized oligonucleotide primer
ggtcggtgtgaacggatttg

SEQ ID NO: 10 - DNA, 22 bp, Artificial Sequence
  Artificially synthesized oligonucleotide primer
ggagtcatactggaacatgtag

SEQ ID NO: 11 - DNA, 28 bp, Artificial Sequence
  Artificially synthesized oligonucleotide primer
gtgtttttggcgattaaacataatcctg

SEQ ID NO: 12 - DNA, 30 bp, Artificial Sequence
  Artificially synthesized oligonucleotide primer
actgactcgtgtaccataatatgttaccag

SEQ ID NO: 13 - DNA, 23 bp, Artificial Sequence
  Artificially synthesized mutagenized oligonucleotide
cgccaaaaacacacuagctcgug

SEQ ID NO: 14 - DNA, 22 bp, Artificial Sequence
  Artificially synthesized Mmu-miR-193a sequence
ugacccugaaacauccggucaa

SEQ ID NO: 15 - DNA, 42 bp, Artificial Sequence
  Artificial mammalian Caprin 1 conserved subsequence
aaaaacacacuggccaguguaccauaauauguuaccagaaga

SEQ ID NO: 16 - DNA, 41 bp, Artificial Sequence
  Artificial chicken Caprin 1 conserved subsequence
aaaaacacacuggccaguguacuauacauguuaccagaaga

SEQ ID NO: 17 - DNA, 15 bp, Artificial Sequence
  Artificial frog Caprin 1 subsequence
uggucaggggcucuu